(19) United States
(12) Patent Application Publication
(10) Pub. No.: US 2024/0301460 A1
(43) Pub. Date: Sep. 12, 2024

(54) ENZYMATIC SYNTHESIS OF 4'-ETHYNYL NUCLEOSIDE ANALOGS

(71) Applicant: Merck Sharp & Dohme LLC, Rahway, NJ (US)

(72) Inventors: Mark A. Huffman, Warren, NJ (US); Anna Fryszkowska, Scotch Plains, NJ (US); Joshua N. Kolev, Califon, NJ (US); Paul N. Devine, Tinton Falls, NJ (US); Kevin R. Campos, Berkeley Heights, NJ (US); Matthew Truppo, Princeton, NJ (US); Christopher C. Nawrat, Garwood, NJ (US)

(73) Assignee: Merck Sharp & Dohme LLC, Rahway, NJ (US)

(21) Appl. No.: 18/675,302

(22) Filed: May 28, 2024

Related U.S. Application Data
(62) Division of application No. 17/257,792, filed on Jan. 4, 2021, now Pat. No. 12,037,623.
(60) Provisional application No. 62/822,320, filed on Mar. 22, 2019.
(60) Provisional application No. 62/695,508, filed on Jul. 9, 2018.

(51) Int. Cl. C12P 19/40; C07C 33/042; C07F 9/113; C07H 13/00; C12P 7/18; C12P 9/00; C12P 17/04

(52) U.S. Cl. CPC C12P 19/40; C07C 33/042; C07F 9/113; C07H 13/00; C12P 7/18; C12P 9/00; C12P 17/04

(57) ABSTRACT

The present invention relates to an enzymatic synthesis of 4′-ethynyl-2′-deoxy nucleosides and analogs thereof, for example EFdA, that eliminates the use of protecting groups on the intermediates, improves the stereoselectivity of glycosylation and reduces the number of process steps needed to make said compounds. It also relates to the novel intermediates employed in the process.

### CROSS-REFERENCE TO RELATED APPLICATIONS

This application is a divisional application of U.S. patent application Ser. No. 17/257,792, filed Jan. 4, 2021, which is a National Stage application of International Patent Application No. PCT/US2019/040316, filed Jul. 2, 2019, which claims priority to U.S. Provisional Patent Application No. 62/695,508, filed Jul. 9, 2018, and U.S. Provisional Patent Application No. 62/822,320, filed Mar. 22, 2019.

### REFERENCE TO SEQUENCE LISTING SUBMITTED ELECTRONICALLY

The sequence listing of the present application is submitted electronically via EFS-Web as an XML sequence listing with a file name “24608USDIV-SEQLIST-01MAY2024.xml”, having a creation date of May 1, 2024, and a size of 27.7 kb. This sequence listing submitted via EFS-Web is part of the specification and is herein incorporated by reference in its entirety.

### BACKGROUND OF THE INVENTION

4′-Ethynyl-2′-deoxy nucleoside analogs are known for activity against HIV, AIDS and related diseases.

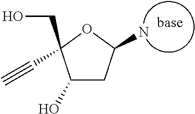

One example of a 4′-ethynyl nucleoside analog is 4′-ethynyl-2-fluoro-2′-deoxyadenosine (EFdA, also known as MK-8591) which is a nucleoside reverse transcriptase translocation inhibitor that blocks HIV-1 and SIV viral replication in vitro (Kawamoto, A.,

Kodama, E., Sarafianos S. F. et al, Int. J. Biochem. Cell Biol.; 40(11):2410-20 [2008]; Ohrui, H., Kohgo, S., Hayakawa, H. et al, Nucleosides, Nucleotides & Nucleic Acids, 26, 1543-1546 [2007]) and in vivo (Hattori, S., Ide, K., Nakata, H. et al. Antimicrobial. Agents and

Chemotherapy, 53, 3887-3893 [2009]). EFdA is claimed in U.S. Pat. No. 7,339,053 (referred to in the '053 patent as 2′-deoxy-4′-C-ethynyl-2-fluoroadenosine). EFdA has the following chemical structure:

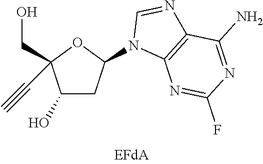 [SMILES: C#C[C@]1(CO)O[C@@H](n2cnc3c(N)nc(F)nc32)C[C@@H]1O]

EFdA is metabolized in cells to its active triphosphate anabolite which inhibits HIV reverse transcriptase. In contrast to nucleoside reverse transcriptase inhibitors (NsRTIs) and nucleotide reverse transcriptase inhibitors (NtRTIs) currently available for the treatment of HIV infection which lack a 3′-OH group to block incorporation of incoming nucleotide, EFdA retains a 3′ OH group and acts as a chain terminator by preventing translocation of the primer:template in the reverse transcriptase (RT) active site and preventing binding of incoming deoxyribonucleotide triphosphates (dNTPs). In addition, the pucker of the modified ribose ring of EFdA is believed to contribute to inhibition of reverse transcriptase by placing the 3′-OH in a vector in which phosphotransfer from the incoming nucleotide is inefficient. (Michailidis E, et al., Mechanism of inhibition of HIV-1 reverse transcriptase by 4′-ethynyl-2-fluoro-2′-deoxyadenosine triphosphate, J Biol Chem 284:35681-35691 [2009]; Michailidis E, et al., 4′-Ethynyl-2-fluoro-2′-deoxyadenosine (EFdA) inhibits HIV-1 reverse transcriptase with multiple mechanisms, J Biol Chem 289:24533-24548).

In in-vitro HIV replication assays, EFdA is a potent antiretroviral and exhibits comparable antiviral activity against clinical isolates across all subtypes that have been evaluated. It is rapidly anabolized in both lymphoid derived cell lines and in peripheral blood mononuclear cells to the active triphosphate in vitro, and the intracellular half-life of EFdA Triphosphate (EFdA-TP) exceeds 72 hrs. (Stoddart, C. A., Galkina, et al., Oral Administration of the Nucleoside EFdA (4′-Ethynyl-2-Fluoro-2′-Deoxyadenosine) Provides Rapid Suppression of HIV Viremia in Humanized Mice and Favorable Pharmacokinetic Properties in Mice and the Rhesus Macaque, Antimicrob Agents Chemother, 2015 July; 59(7): 4190-4198, Published online 2015 May 4).

EFdA has been shown to have efficacy in animal models of HIV infection including humanized mouse models and an SIV infected rhesus macaque model. Pharmacokinetic studies of orally administered EFdA in mouse and rhesus monkey have demonstrated rapid absorption and high plasma concentrations. A long intracellular half-life was demonstrated by the fact that isolated peripheral blood mononuclear cells from the rhesus macaque were refractory to SIV infection 24 hr after drug administration. (Ibid.)

Previous syntheses of 4′-ethynyl nucleoside analogs including EFdA suffer from modest stereoselectivity in the formation of the C—N bond between the ethynyl-deoxyribose sugar and the 2-fluoroadenine (also referred to as 2-fluoro-9H-purin-6-amine) nucleobase. The previous syntheses also require protecting groups to carry out the glycosylation reaction which reduces the efficiency of the syntheses.

The synthesis described in Kei Fukuyama, et al., Synthesis of EFdA via a Diastereoselective Aldol Reaction of a Protected 3-Keto Furanose, Organic Letters 2015, 17(4), pp. 828-831; DOI: 10.1021/015036535) is a 14-step synthesis from D-glucose diacetonide that uses diastereoselective reactions to set the three stereocenters. The stereochemistry of the anomeric center is controlled by having a 2′-acetoxy directing group that is subsequently removed by hydrolysis and deoxygenation. This route requires 4 chromatographic purifications, and the stoichiometric use of a toxic organotin reagent for late-stage deoxygenation.

In another route (see Mark McLaughlin, et al., Enantioselective Synthesis of 4′-Ethynyl-2-fluoro-2′-deoxyadenosine (EFdA) via Enzymatic Desymmetrization, Organic Letters 2017, 19 (4), pp. 926-929), the fully-substituted 4′-carbinol is generated stereoselectively with an enzymatic desymmetrization. The 3′-stereocenter is set with a catalytic asymmetric transfer hydrogenation, and the anomeric l′-linkage is established in modest stereoselectivity using substrate control, with an upgrade in stereochemical purity achieved by crystallization of an intermediate. This process requires 15 steps, requires the use of several protecting groups and generates the glycosyl linkage between the nucleobase and sugar fragments in low stereoselectivity (1.8:1).

A 12-step synthesis for making EFdA from R-glyceraldehyde acetonide is described in Kageyama, M., et al., Concise Synthesis of the Anti-HIV Nucleoside EFdA, Biosci. Biotechnol. Biochem, 2012 , 76, pp. 1219-1225; and Enantioselective Total Synthesis of the Potent Anti-HIV Nucleoside EFdA, Masayuki Kageyama, et al., Organic Letters 2011 13 (19), pp. 5264-5266 [DOI: 10.1021/ol202116k]. The syntheses use the chiral starting material to set the 3′-stereocenter with moderate diastereoselectivity. After chromatographic separation of stereoisomers, the new stereocenter is used to guide a diastereoselective alkyne addition to set the fully-substituted 4′-stereocenter. The anomeric l′-position is established with little stereocontrol and requires chromatography to separate the anomers. This route requires chromatographic separation of diastereoisomers at two different stages and starts from an expensive chiral starting material.

Kohgo, S., et al., Design, Efficient Synthesis, and Anti-HIV Activity of 4′-C-Cyano- and 4′-C-Ethynyl-2′-deoxy Purine Nucleosides, Nucleosides, Nucleotides and Nucleic Acids, 2004, 23, pp. 671-690 [ DOI: 10.1081/NCN-120037508] describes a synthetic route that starts from an existing nucleoside and modifies both the sugar and nucleobase portions. It is an 18-step synthesis starting from 2-amino-2′-deoxyadenosine with a low 2.5% overall yield.

It is known that enzymes such as purine nucleoside phosphorylase (PNP, EC 2.4.2.1) can form the glycosyl linkage in nucleosides and nucleoside analogs in high stereoselectivity and without the use of protecting groups. See for example the review: New Trends in Nucleoside Biotechnology, Mikhailopulo, I. A., Miroshnikov, A. I,. Acta Naturae 2010, 2, pp. 36-58. However, the current scope of the sugar fragments capable of undergoing reaction catalyzed by PNP has been limited to the α-1-phosphates of natural ribose and deoxyribose along with a small number of analogs with small H, NH₂, or F substituents at the C2′ and C3′ positions and replacements of the C5′ OH group. There have been no reports of successful glycosylation catalyzed by PNP using sugars with carbon substituents on the ring or any substitution at the C4′ position.

Access to the ribose and deoxyribose α-1-phosphate substrates for the PNP-catalyzed glycosylation has been demonstrated by translocation of the phosphate group from the 5′-hydroxyl to l′-hydroxyl position with the enzyme phosphopentomutase (PPM, EC 5.4.2.7) (sec Mikhailopulo, I. A., et al. supra). However, the scope of the sugars for which PPM is capable of catalyzing this reaction has been limited to ribose, arabinose, 2-deoxyribose, and 2,3-dideoxyribose. No examples have been reported of successful reaction with sugar phosphates containing any additional substituents.

Deoxyribose phosphate aldolase (DERA, EC 4.1.2.4) enzymes are known to catalyze the aldol addition of acetaldehyde to other short-chain aldehydes (see review: Stephen M. Dean, et al., Recent Advances in Aldolase-Catalyzed Asymmetric Synthesis, Adv. Synth. Catal. 2007, 349, pp. 1308-1320; DOI: 10.1002/adsc.200700115). However, no examples have been reported with aldehydes bearing a fully substituted carbon a to the aldehyde.

U.S. Pat. No. 7,229, 797 describes the formation of deoxyribonucleosides from the natural unsubstituted deoxyribose 1-phosphate by use of purine nucleoside phosphorylase (PNP) and additionally using enzymes such as sucrose phosphorylase to remove the inorganic phosphate byproduct and drive the equilibrium. It does not disclose enzyme engineering for the creation of PNP enzymes that can generate nucleosides from the unnatural 4-ethynyl-D-2-deoxyribose 1-phosphate, nor that through engineering of PPM and DERA enzymes to act on unnatural substrates, 4-ethynyl-D-2-deoxyribose 1-phosphate can be generated.

In view of the difficult and lengthy synthetic options developed to date for producing 4′-ethynyl nucleoside analogs, it would be desirable to develop an improved enzymatic synthesis for 4′-ethynyl nucleoside analogs such as EFdA that reduces the number of process steps, minimizes the use of protecting groups, improves the stereoselectivity of glycosylation and avoids the use of toxic materials.

Surprisingly, it has been found that PPM enzymes have some activity with the 3-atom ethynyl substituent at the 4′ position on ribose and that the PPM enzyme activity could be improved by introducing mutations into the enzymes to successfully develop a reaction for isomerization of 4-ethynyl-D-2-deoxyribose 5-phosphate (6) to 4-ethynyl-D-2-deoxyribose 1-phosphate (6.5) catalyzed by PPM to enable a more efficient method for production of 4′-ethynyl-2′-deoxy nucleosides.

Additionally, PNP enzymes have also been found to have some activity with the 3-atom ethynyl substituent at the 4 position on deoxyribose and that the PNP enzyme activity could be improved by introducing mutations into the enzymes to successfully develop a glycosylation reaction catalyzed by PNP to enable a more efficient method for production of 4′-ethynyl-2′-deoxy nucleosides.

Even further improvement to the overall synthetic method came from the finding that DERA enzymes, particularly the DERA from Shewanella halifaxensis, have activity for aldol reaction with 2-ethynyl-glyceraldehyde 3-phosphate which has a fully substituted α-carbon. This discovery allowed for the efficient synthesis of 4-ethynyl-D-2-deoxyribose 5-phosphate, a precursor to 4′-ethynyl-2′-deoxy nucleoside analogs, e.g., including EFdA.

### SUMMARY OF THE INVENTION

The present invention involves the use of engineered enzymes in a novel enzymatic synthesis of 4′-ethynyl-2′-deoxy nucleoside analogs, including EFdA, that eliminates the use of protecting groups on intermediates, improves the stereoselectivity of glycosylation and greatly reduces the number of process steps needed to make said compounds compared to prior methods, among other process improvements. It further relates to novel intermediates which are an integral part of the enzymatic process.

The overall process is summarized in the following Scheme 1 and Scheme 2; the latter scheme provides an alternative method for making compound 5:

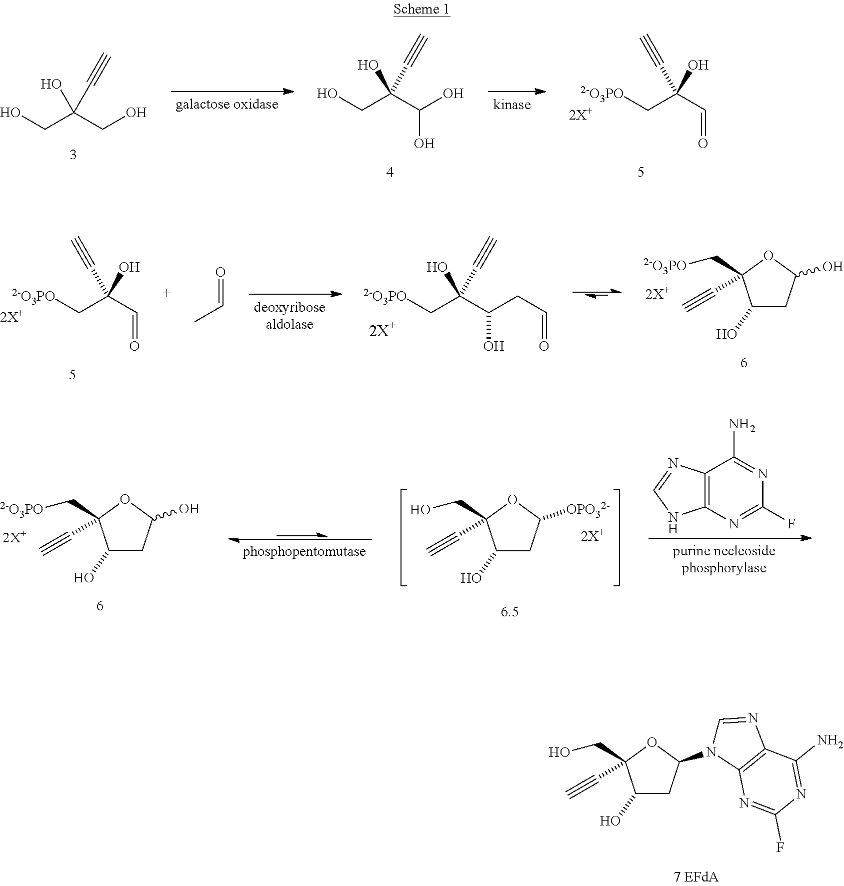

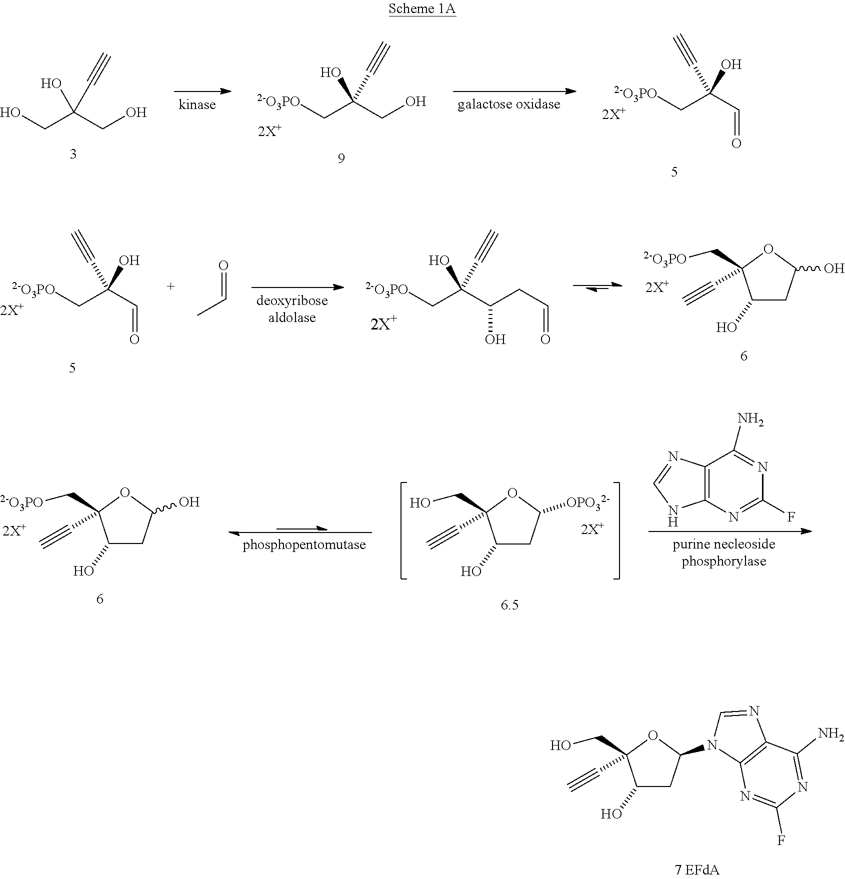

The acid form or salts of phosphate intermediates can be employed in the process described herein and are not limited to specific acid or salt forms provided in exemplifications of the process steps herein. For all phosphate intermediates described herein, 2X+ represents any combination of two protons, one proton with one other monovalent cation, two monovalent cations (the same or different) or one divalent cation. Phosphate intermediates drawn herein with-HO₃PO-likewise can have any combination of two protons, one proton with one other monovalent cation, two monovalent cations (the same or different) or one divalent cation, associated with the phosphate group. Examples include, but are not limited to, salts of calcium, magnesium, or zinc; mono or di-sodium salts, mono or di-potassium salts, mono or di-lithium salts; mono or di-ammonium salts; or mono-or di-valent salts with primary, secondary or tertiary amines.

As is well understood in the art, the intermediate compounds shown or named herein as aldehyde or hydrate in the synthetic steps herein can exist in either form or a mixture of such forms in the reactions described herein. For example, compounds (4) and (5) are depicted in Scheme 1 as a hydrate and an aldehyde, respectively, but each can exist in hydrate or aldehyde form or a mixture thereof in the reaction steps where each is present. Each such form is encompassed by reference to compound numbers (4) or (5) within the process steps herein:

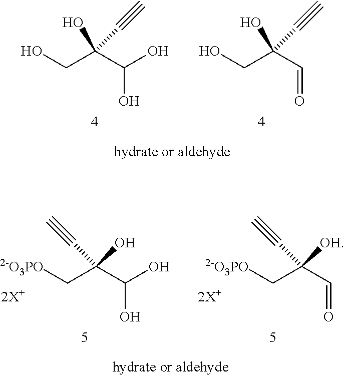

Compound (3) is achiral and may be shown herein as either of the following:

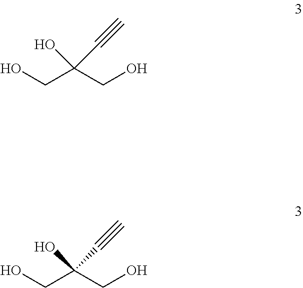

Compound (6) can exist in its ring form or as an open chain aldehyde or hydrate, each as an acid or a salt thereof, in the reaction steps where it is present:

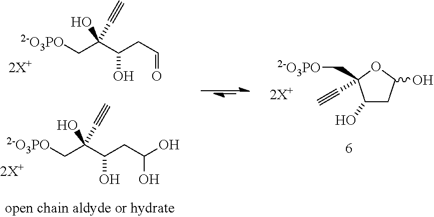

### DETAILED DESCRIPTION OF THE INVENTION

4′-Ethynyl-2′-deoxy nucleosides and analogs thereof

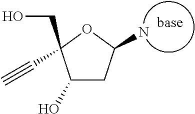

having an anomeric C—N linkage have been explored for activity against HIV, AIDS and related diseases. 4′-Ethynyl-2′-deoxy nucleosides and analogs thereof comprise a 4′-ethynyl-2′-deoxy ribose attached via an anomeric C-N linkage to a purine or pyrimidine nucleobase (adenine, guanine, cytosine, thymine or uracil) or a modified purine or pyrimidine nucleobase.

It has been discovered that 4′-ethynyl-2′-deoxy nucleoside analogs such as EFdA can be synthesized employing a final step one-pot process by combining 4-ethynyl-D-2-deoxyribose 5-phosphate (6) with two enzymes, phosphopentomutase (PPM) [for example but not limited to SEQ ID NO.: 8] and purine nucleoside phosphorylase (PNP) [for example but not limited to SEQ ID NO.: 9, SEQ ID NO.: 15], as shown in Scheme 2.

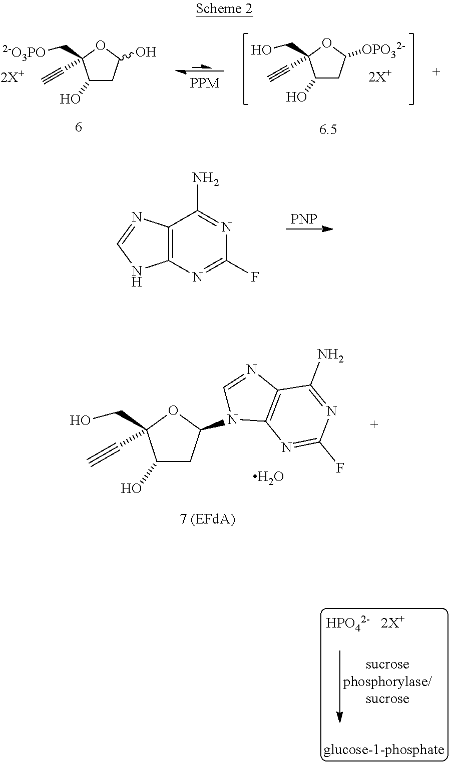

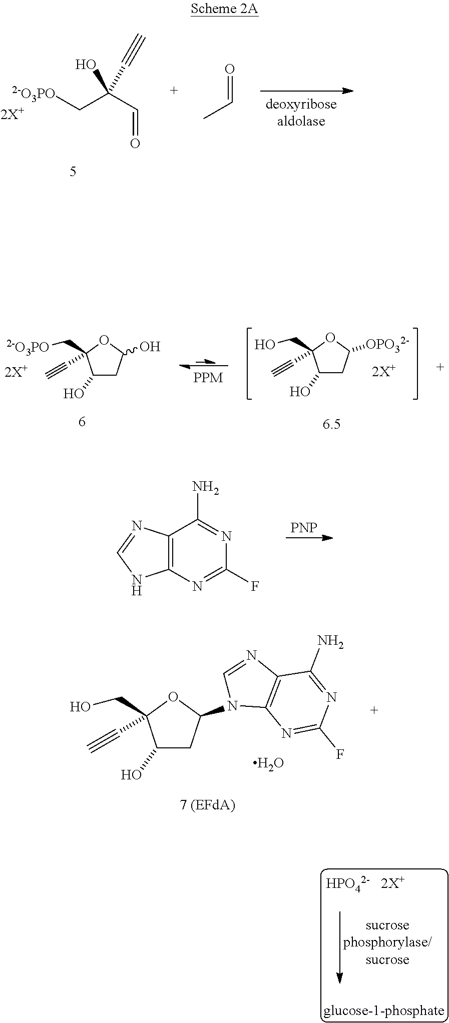

As shown in Scheme 2, the final step of the synthesis employs a 2-enzyme reaction with an optional 3^{rd} enzyme to drive the equilibrium of the reaction toward the desired end product. The final step starts with compound (6) or a salt thereof wherein (6) is 4-ethynyl-2-deoxyribose 5-phosphate in ring form as shown above or the open chain aldehyde or hydrate form thereof.

Compound (6) is combined with phosphopentomutase (PPM), purine nucleoside phosphorylase (PNP), sucrose phosphorylase, sucrose, and a nucleobase e.g., unsubstituted or substituted adenine, in a buffered solution containing a manganese (II) salt and adjusted as needed to a pH in a range from about 6.5 to 8.0, or more particularly from about 7.0 to 7.5. A molar ratio of sucrose:compound (6) can be, but is not limited to, from about 1:1 to 4:1. The components of this one-pot reaction can be combined in any order.

The reaction is agitated within a temperature range that does not denature the enzymes, e.g., from about 30 to 45° C., and more particularly from about 35 to 45° C. Up to a certain point, cooler temperatures may work but will slow the reaction rate.

Any buffer with a suitable pH and containing a manganese (II) salt may be used in the reaction. Examples of such buffers include but are not limited to: triethanolamine; PIPES, e.g. piperazine-N,N′-bis(2-ethanesulfonic acid); MOPS, e.g., 3-(N-morpholino)propanesulfonic acid or 3-morpholinopropane-1-sulfonic acid; HEPES, e.g., 4-(2-hydroxyethyl)-1-piperazineethanesulfonic acid or 2-[4-(2-hydroxyethyl)piperazin-1-yl]ethanesulfonic acid; TRIS, e.g., tris(hydroxymethyl)aminomethane or 2-Amino-2-(hydroxymethyl)propane-1,3-diol; and BIS-TRIS methane, e.g., 2-[Bis(2-hydroxyethyl)amino]-2-(hydroxymethyl)propane-1,3-diol. More particularly, the buffer is triethanolamine. The manganese (II) salt in the buffer can be, for example, manganese chloride, manganese chloride hydrate, manganese bromide, manganese iodide, manganese nitrate, and/or manganese sulfate. The manganese concentration in the buffer can range from about 0.05 mM to about 10 mM, and particularly it is about 5 mM.

The equilibrium reaction can be driven forward to high conversion of the final product by consumption of the byproduct inorganic phosphate salt by phosphorolysis of sucrose to D-fructose and α-D-glucose-1-phosphate, catalyzed by sucrose phosphorylase (EC 2.4.1.7) added to the reaction mixture. However, any other options for removing phosphate during the reaction can be employed, e.g., adding calcium, magnesium, or manganese to the reaction to precipitate a phosphate salt instead of using sucrose phosphorylase and sucrose. This highly efficient and ecologically friendly process has the advantage of forming the anomeric linkage between sugar and nucleobase with very high stereoselectivity without the use of protecting groups or organic solvents and can be performed as a one pot reaction.

Once the reaction is complete, the final product can be isolated using standard procedures known to persons of ordinary skill in the art, such as but not limited to, isolation by crystallization of the final product and collection by filtration, or extraction into an appropriate solvent followed by crystallization.

As shown in Scheme 2A, the final step of the synthesis can alternatively employ a 3-enzyme reaction with an optional 4^{th} enzyme to drive the equilibrium of the reaction toward the desired end product. The final step starts with compound (5) or a salt thereof, wherein (5) is (R)-2-ethynyl-glyceraldehyde 3-phosphate or a hydrate form thereof.

Compound (5) is combined with deoxyribose-phosphate aldolase (DERA), acetaldehyde, phosphopentomutase (PPM), purine nucleoside phosphorylase (PNP), sucrose phosphorylase, sucrose, and a nucleobase or an analog thereof e.g., unsubstituted or substituted adenine, in a buffered solution containing a manganese (II) salt and adjusted as needed to a pH in a range from about 4 to 10, or particularly from about 6.5 to 8.0, or more particularly from about 7.0 to 7.5. A molar ratio of sucrose:compound (5) can be, but is not limited to, from about 1:1 to 4:1. The components of this one-pot reaction can be combined in any order.

The reaction is performed within a temperature range that does not denature the enzymes, for example from about 30 to 45° C., or particularly from about 35 to 45° C. Up to a certain point, cooler temperatures may work but will slow the reaction rate.

The acetaldehyde is added as a solution, and more particularly as a 40 wt % solution in isopropyl alcohol. Any suitable solution of acetaldehyde or neat acetaldehyde may be used in the reaction. Examples of such solutions include but are not limited to: acetaldehyde solution in isopropanol, acetaldehyde solution in ethanol, acetaldehyde solution in water, acetaldehyde solution in THF. A molar ratio of aldehyde:compound (5) can be, but is not limited to, from about 0.5:1 to 4:1, and more particularly 1.5:1.

Any buffer with a suitable pH and containing a manganese (II) salt may be used in the reaction. Examples of such buffers include but are not limited to: triethanolamine; PIPES, e.g. piperazine-N,N′-bis(2-ethanesulfonic acid); MOPS, e.g., 3-(N-morpholino)propanesulfonic acid or 3-morpholinopropane-1-sulfonic acid; HEPES, e.g., 4-(2-hydroxyethyl)-1-piperazineethanesulfonic acid or 2-[4-(2-hydroxyethyl)piperazin-1-yl]ethanesulfonic acid; TRIS, e.g., tris(hydroxymethyl)aminomethane or 2-Amino-2-(hydroxymethyl)propane-1,3-diol; and BIS-TRIS methane, e.g., 2-[ Bis(2-hydroxyethyl)amino]-2-(hydroxymethyl)propane-1,3-diol. More particularly, the buffer is triethanolamine. The manganese (II) salt in the buffer can be, for example, manganese chloride, manganese chloride hydrate, manganese bromide, manganese iodide, manganese nitrate, and/or manganese sulfate. The manganese concentration in the buffer can range from about 0.05 mM to about 10 mM, and particularly it is about 5 mM.

The equilibrium reaction can be driven forward to high conversion of the final product by consumption of the byproduct inorganic phosphate salt by phosphorolysis of sucrose to D-fructose and α-D-glucose-1-phosphate, catalyzed by sucrose phosphorylase (EC 2.4.1.7) added to the reaction mixture. However, any other options for removing phosphate during the reaction can be employed, e.g., adding calcium, magnesium, or manganese to the reaction to precipitate a phosphate salt instead of using sucrose phosphorylase and sucrose. This highly efficient and ecologically friendly process has the advantage of forming the anomeric linkage between sugar and nucleobase with very high stereoselectivity without the use of protecting groups or organic solvents and can be performed as a one pot reaction.

Once the reaction is complete, the final product can be isolated using standard

procedures known to persons of ordinary skill in the art, such as but not limited to, isolation by crystallization of the final product and collection by filtration, or extraction into an appropriate solvent followed by crystallization.

Several upstream intermediates used in the present process for the synthesis of the final product 4′-ethynyl-2′-deoxy nucleosides and analogs thereof are also made using enzymatic reaction methods as shown in Scheme 3; Scheme 3A and Scheme 3B

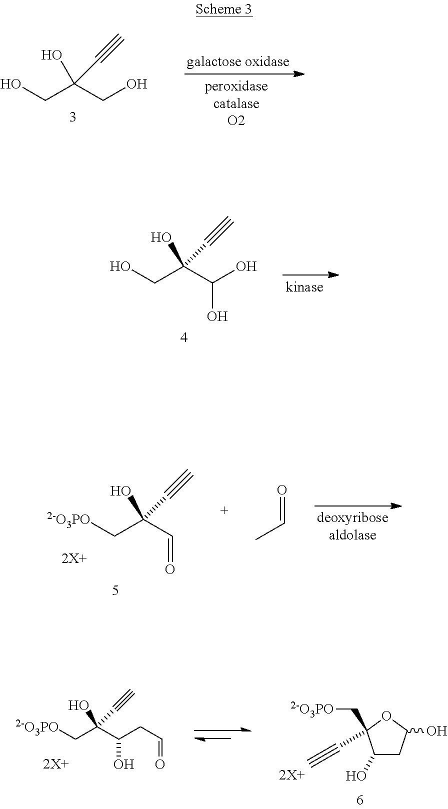

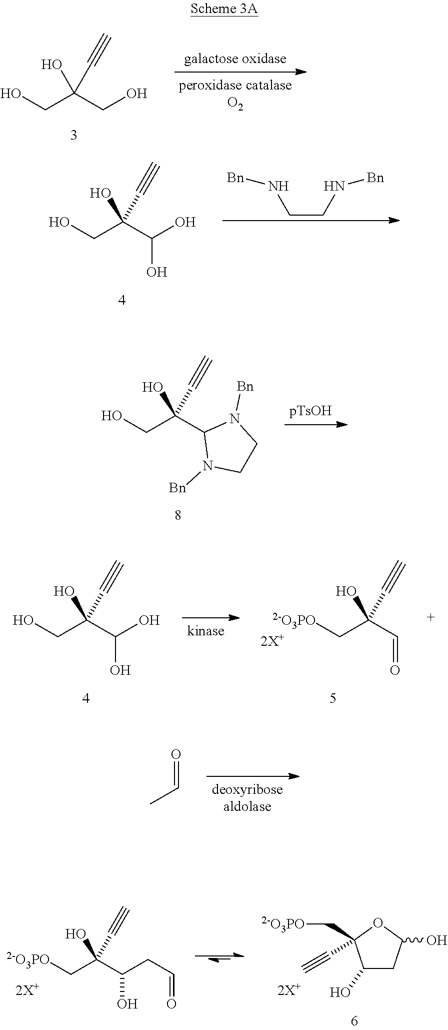

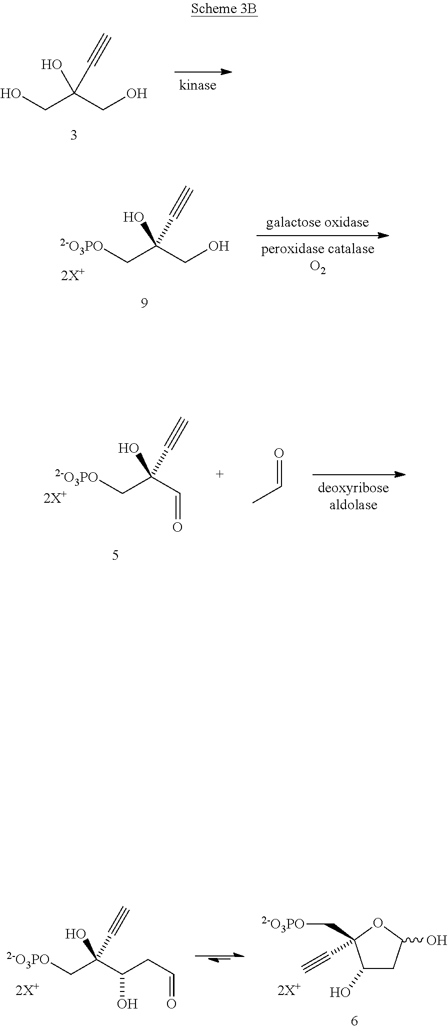

### Compound 4: Oxidase Reaction

As shown in Scheme 3, (R)-2-ethynyl-glyceraldehyde (4) is prepared by reacting galactose oxidase with 2-ethynyl-propane-1,2,3-triol (3) in a buffered solution adjusted as needed to a pH in a range from about 3 to 10, or more particularly from about 6 to 8. Any buffer having a suitable pH range can be used, for example but not limited to, sodium phosphate; sodium acetate; PIPES, e.g. piperazine-N,N′-bis(2-ethanesulfonic acid); MOPS, e.g., 3-(N-morpholino)propanesulfonic acid or 3-morpholinopropane-1-sulfonic acid; HEPES, e.g., 4-(2-hydroxyethyl)-1-piperazineethanesulfonic acid or 2-[4-(2-hydroxyethyl)piperazin-1-yl]ethanesulfonic acid; TRIS, e.g., tris(hydroxymethyl)aminomethane or 2-Amino-2-(hydroxymethyl)propane-1,3-diol; and BIS-TRIS methane, e.g., 2-[Bis(2-hydroxyethyl)amino]-2-(hydroxymethyl)propane-1,3-diol; borate; CAPS, e.g., N-cyclohexyl-3-aminopropanesulfonic acid,; MES, e.g. 2-(N-morpholino)ethanesulfonic acid; CHES, e.g., N-Cyclohexyl-2-aminoethanesulfonic acid; Glycine; or Bicine (N,N-Bis(2-hydroxyethyl)glycine); with sodium phosphate being preferred.

Copper and a peroxidase are both used in the reaction to activate galactose oxidase (GOase). Copper can be supplied to the reaction mixture by addition of CuSO4, Cu(OAc)2, CuCl₂ or other salts of Cu(II) or Cu(I). The peroxidase can be a horseradish peroxidase, or a peroxidase derived from other organisms, or it can be replaced by an oxidant such as ferricyanide, iridate, manganese (III) salts, persulfate salts and other one electron or two electron oxidants, or inorganic or organic oxidants. Preferably, the peroxidase is a horseradish peroxidase. A catalase is also added to help prevent GOase deactivation. The catalase can be from a mammalian source (bovine) or from a bacterial or fungal source such as Corynebacterium, Aspergillus or other organisms known in the art for this purpose.

The reaction proceeds in the presence of oxygen. One convenient method is sparging the reaction with air. Alternatively, other systems to generate oxygen can employed, such as hydrogen peroxide/catalase, superoxide or use of other methods known in the art for this purpose.

The reaction can be performed with a substrate concentration of about 10 to 180 g/L, and particularly 20 to 50 g/L. The reaction can be run at a temperature from about 0 to 40° C., and particularly from about 10 to 30° C.

### Compound 8: Aminal Formation

As exemplified in Scheme 3A, (R)-2-ethynyl-glyceraldehyde (4) can be isolated in its aminal form (for example, compound 8) by reacting it with any amine, diamine or amino alcohol that forms a stable N,N-acetal or N,O-acetal, for example but not limited to, N,N′-dibenzylethane-1,2-diamine, N,N′-dimethylethane-1,2-diamine, N,N′-diphenylethane-1,2-diamine, and N-benzylethanolamine; with N,N′-dibenzylethane-1,2-diamine being preferred. The reaction is performed in an organic solvent at a temperature at or below about 50° C., preferably from 20 to 30° C., to avoid the decomposition of the aminal. Any solvent that is not miscible with water can be used, for example but not limited to, MTBE, 2-MeTHF, CPME, diethyl ether, diisopropyl ether, ethyl acetate, isopropyl acetate, toluene, DCM or a mixture thereof, with MTBE being preferred. The reaction can be performed with a substrate concentration of about 10 to 100 g/L, and particularly 20 to 50 g/L.

Optionally the aminal can be further purified by crystallization from an organic

solvent, for example but not limited to, MTBE, 2-MeTHF, CPME, diethyl ether, diisopropyl ether, ethyl acetate, isopropyl acetate, toluene, DCM or a mixture thereof, with MTBE being preferred. The crystallization is performed at or below 50° C., for example at about 40° C., to avoid the decomposition of the aminal.

The reaction proceeds in the absence of oxygen. One convenient method is sparging the reaction with N₂. Alternatively, other systems to exclude oxygen can employed, such as argon, helium, or use of other methods known in the art for this purpose.

Compound 4: Aldehyde 4 Regeneration from the Aminal 8

(R)-2-Ethynyl-glyceraldehyde (4) can be regenerated from its respective aminal by reacting it with an organic or inorganic acid in the presence of organic solvent that is not miscible with water, at a temperature at or below 50° C., for example from about 0 to 15° C., to avoid the decomposition of the aminal. Any organic or inorganic acid can be used, for example but not limited to, p-toluenesulfonic acid, methanesulfonic acid, camphoresulfonic acid, acetic acid, hydrochloric acid, phosphoric acid, sulphuric acid. p-Toluenesulfonic acid is preferred in the reaction with aminal 8 due to low solubility of the N,N′-dibenzylethane-1,2-diamine bis p-toluenesulfonate salt in water. Any solvent that is not miscible with water can be used, for example but not limited to, MTBE, 2-MeTHF, CPME, diethyl ether, diisopropyl ether, ethyl acetate, isopropyl acetate, toluene, DCM or a mixture thereof; with MTBE and 2-MeTHF being preferred. The reaction can be performed with a substrate concentration of about 5 to 100 g/L, and particularly 20 to 50 g/L.

Optionally the aldehyde 4 solution can be further treated with a resin to remove the excess of the organic or inorganic acid. The resin treatment can be performed with basic resins such as DOWEX™ MARATHON™ A resin (hydroxide form) and AMBERLYST® 15 resin (hydrogen form), or the mixture thereof, preferably a mixture DOWEX™ MARATHON™ A resin (hydroxide form) and AMBERLYST® 15 resin.

Optionally the aldehyde 4 solution can be further evaporated under vacuum or swept with a gas to remove the excess of organic solvent.

### Compound 5: Kinase Reaction

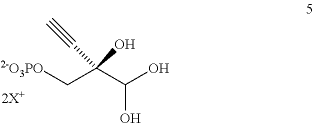

As shown in Scheme 3 and Scheme 3A, (R)-2-ethynyl-glyceraldehyde 3-phosphate hydrate (5) is prepared by reacting pantothenate kinase (PanK) wild type from E. coli or a variant thereof, with compound (4) in a buffered solution adjusted as needed to a pH in a range from about 4 to 10, or particularly about 6.5 to 8.5 or more particularly 5.5 to 8.5 Any buffer having a suitable pH range can be used, for example but not limited to, sodium phosphate, PIPES, e.g. piperazine-N,N′-bis(2-ethanesulfonic acid); BIS-TRIS methane, e.g., 2-[Bis(2-hydroxyethyl)amino]-2-(hydroxymethyl)propane-1,3-diol; borate; HEPES, e.g., 4-(2-hydroxyethyl)-1-piperazinethanesulfonic acid or 2-[4-(2-hydroxyethyl)piperazin-1-yl]ethanesulfonic acid; triethanolamine and TRIS, e.g., TRIS, e.g., tris(hydroxymethyl)aminomethane or 2-Amino-2-(hydroxymethyl)propane-1,3-diol; with sodium phosphate being preferred. The reaction can be performed in the presence of any suitable bi-valent metal salt, for example but not limited to a magnesium salt, for example magnesium chloride, and salts of cobalt, manganese, zinc or calcium.

This reaction utilizes adenosine 5′-diphosphate (ADP) as the phosphate source which requires regenerating to 5′-triphosphate (ATP). ATP can be generated in situ and subsequently regenerated by any method known in the art from ADP, adenosine 5′-monophosphate (AMP) or adenosine. For example, a combination of acetyl phosphate together with acetate kinase can be used for regenerating ADP to ATP. For example, in the presence of pyruvate, phosphate and oxygen, a combination of pyruvate oxidase and catalase generates acetyl phosphate, and therefore in the presence of acetate kinase, can be used for regenerating ADP to ATP.

The reaction can be performed with a substrate concentration of about 10 to 100 g/L, and particularly about 20 to 40 g/L. The reaction can be run at a temperature from about 0 to 40° C., and particularly at about 10 to 25° C.

The reaction can also be performed with pantothenate kinase (PanK) immobilized on a resin, or with both PanK and acetate kinase immobilized on the resin. Any suitable enzyme immobilization method known in the art can be used, for example but not limited to, Immobilized Metal-Ion Affinity Chromatography (IMAC) resin, or an affinity resin-immobilization using other biological tags, co-valent immobilization, immobilization on ionic resins, immobilization by adsorption, encapsulation, and/or crosslinked enzymes. For example, the Metal-Ion Affinity Chromatography (IMAC) resin can be used, or any suitable combination of IMAC resin and bi-valent cation can be used wherein the cation can be, for example but not limited to, nickel, cobalt, copper, zinc, iron, and/or aluminum. Particularly, IMAC resin charged with nickel can be used. Preferably, both acetate kinase and pantothenate kinase (PanK) are immobilized on the resin.

### Compound 9: Kinase Reaction

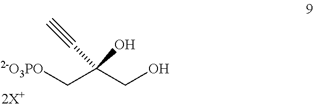

As shown in Scheme 3B, (S)-2-ethynyl-propane-1,2,3-triol 1-phosphate (9) is prepared by reacting pantothenate kinase (PanK) wild type from E. coli or a variant thereof, with compound (3) in a buffered solution adjusted as needed to a pH in a range from about 4 to 10, or particularly about 6.5 to 8.5 or more particularly 5.5 to 8.5 Any buffer having a suitable pH range can be used, for example but not limited to, sodium phosphate, PIPES, e.g. piperazine-N,N′-bis(2-ethanesulfonic acid); BIS-TRIS methane, e.g., 2-[Bis(2-hydroxyethyl)amino]-2-(hydroxymethyl)propane-1,3-diol; borate; HEPES, e.g., 4-(2-hydroxyethyl)-1-piperazineethanesulfonic acid or 2-[4-(2-hydroxyethyl)piperazin-1-yl]ethanesulfonic acid;, triethanolamine and TRIS, e.g., tris(hydroxymethyl)aminomethane or 2-Amino-2-(hydroxymethyl)propane-1,3-diol, with sodium phosphate being preferred. The reaction can be performed in the presence of any suitable bi-valent metal salt, for example but not limited to a magnesium salt, for example magnesium chloride, and salts of cobalt, manganese, zinc or calcium.

This reaction utilizes adenosine 5′-diphosphate (ADP) as the phosphate source which requires regenerating to 5′-triphosphate (ATP). ATP can be generated in situ and subsequently regenerated by any method known in the art from ADP, adenosine 5′-monophosphate (AMP) or adenosine. For example, a combination of acetyl phosphate together with acetate kinase can be used for regenerating ADP to ATP. Alternatively, (a) a combination of pyruvate oxidase, catalase and acetate kinase in the presence of pyruvate, phosphate and oxygen can be used for regenerating ADP to ATP, or (b) a combination of pyruvate oxidase, catalase and acetate kinase in the presence of pyruvate, phosphate, and oxygen in combination with acetyl phosphate and acetate kinase can be used for ATP regeneration from ADP.

The reaction can be performed with a substrate concentration of about 10 to 100 g/L, and particularly about 20 to 40 g/L. The reaction can be run at a temperature from about 0 to 40° C., and particularly at about 10 to 25° C.

The reaction can also be performed with pantothenate kinase (PanK) immobilized on a resin, or with both PanK and acetate kinase immobilized on the resin. Any suitable enzyme immobilization method known in the art can be used, for example but not limited to, Immobilized Metal-Ion Affinity Chromatography (IMAC) resin, or an affinity resin-immobilization using other biological tags, co-valent immobilization, immobilization on ionic resins, immobilization by adsorption, encapsulation, and/or crosslinked enzymes. For example, the Metal-Ion Affinity Chromatography (IMAC) resin can be used, or any suitable combination of IMAC resin and bi-valent cation can be used wherein the cation can be, for example but not limited to, nickel, cobalt, copper, zinc, iron, and/or aluminum. Particularly, IMAC resin charged with nickel can be used. Preferably, both acetate kinase and pantothenate kinase (PanK) are immobilized on the resin.

### Compound 5: Oxidase Reaction

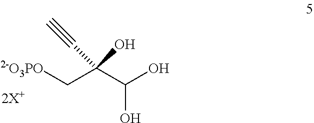

As shown in Scheme 3B, (R)-2-ethynyl-glyceraldehyde hydrate 3-phosphate (5) is prepared by reacting galactose oxidase with (S)-2-ethynyl-propane-1,2,3-triol 1-phosphate (9) in a buffered solution adjusted as needed to a pH in a range from about 3 to 10, or more particularly from about 6 to 8. Any buffer having a suitable pH range can be used, for example but not limited to, sodium phosphate; sodium acetate; PIPES, e.g. piperazine-N,N′-bis(2-ethanesulfonic acid); MOPS, e.g., 3-(N-morpholino)propanesulfonic acid or 3-morpholinopropane-1-sulfonic acid; HEPES, e.g., 4-(2-hydroxyethyl)-1-piperazineethanesulfonic acid or 2-[4-(2-hydroxyethyl)piperazin-1-yl]ethanesulfonic acid; TRIS, e.g., tris(hydroxymethyl)aminomethane or 2-Amino-2-(hydroxymethyl)propane-1,3-diol; and BIS-TRIS methane, e.g., 2-[Bis(2-hydroxyethyl)amino]-2-(hydroxymethyl)propane-1,3-diol; borate; CAPS, e.g., N-cyclohexyl-3-aminopropanesulfonic acid,; MES, e.g. 2-(N-morpholino)ethanesulfonic acid; CHES, e.g., N-Cyclohexyl-2-aminoethanesulfonic acid; Glycine; or Bicine (N,N-Bis(2-hydroxyethyl)glycine); with sodium phosphate being preferred.

Copper and a peroxidase are both used in the reaction to activate galactose oxidase (GOase). Copper can be supplied to the reaction mixture by addition of CuSO₄, Cu(OAc)₂, CuCl₂ or other salts of Cu(II) or Cu(I). The peroxidase can be a horseradish peroxidase, or a peroxidase derived from other organisms, or it can replaced by an oxidant such as ferricyanide, iridate, manganese (III) salts, persulfate salts and other one electron or two electron oxidants, or inorganic or organic oxidants. Preferably, the peroxidase is a horseradish peroxidase. A catalase is also added to help prevent GOase deactivation. The catalase can be from a mammalian source (bovine) or from a bacterial or fungal source such as Corynebacterium, Aspergillus or other organisms known in the art for this purpose.

The reaction proceeds in the presence of oxygen. One convenient method is sparging the reaction with air. Alternatively, other systems to generate oxygen can employed, such as hydrogen peroxide/catalase, superoxide or use of other methods known in the art for this purpose.

The reaction can be performed with a substrate concentration of about 10 to 180 g/L, and particularly 20 to 50 g/L. The reaction can be run at a temperature from about 0 to 40° C., and particularly from about 10 to 30° C.

### Compound 6: Deoxyribose-Phosphate Aldolase (DERA) Reaction

An important advantage of this new route for producing compound (6) over prior known processes is that it creates the sugar framework at the correct oxidation state without the use of protecting groups.

4-Ethynyl-D-2-deoxyribose 5-phosphate (6) is prepared by reacting deoxyribose-phosphate aldolase (DERA) with (R)-2-ethynyl-glyceraldehyde 3-phosphate (5) as an acid or salt thereof, and acetaldehyde in an aqueous solution adjusted as needed to a pH in a range from about 5 to 9, or more particularly about 6 to 8. Examples of salts of (5) include, but are not limited to, calcium, magnesium, zinc, mono-or di-Na salts, mono-or di-K salts, or mono-or di-Li salts; mono-or di-ammonium or salts; or mono-valent or di-valent salts with primary, secondary or tertiary amines. The reaction can be performed in an open vessel or is preferably performed in a sealed vessel to prevent evaporation of acetaldehyde.

The reaction can be performed with a substrate concentration of about 10 to 100 g/L, particularly about 30 to 60 g/L. It can be run at a temperature from about 0 to 40° C., and particularly from about 25 to 35° C.

The reaction can be run without any buffers. Alternatively, buffers can be used, for example but not limited to, triethanolamine; phosphate; MOPS, e.g., 3-(N-morpholino)propanesulfonic acid or 3-morpholinopropane-1-sulfonic acid; HEPES, e.g., 4-(2-hydroxyethyl)-1-piperazineethanesulfonic acid or 2-[4-(2-hydroxyethyl)piperazin-1-yl]ethanesulfonic acid; BIS-TRIS methane, e.g., 2-[Bis(2-hydroxyethyl)amino]-2-(hydroxymethyl)propane-1,3-diol; borate; PIPES, e.g. piperazine-N,N′-bis(2-ethanesulfonic acid); MES, e.g., 2-(N-morpholino)ethanesulfonic acid; and borate; or other buffers having a suitable pH range which do not have any primary amine groups.

Each step and method of the processes described herein which comprise the use of one or more enzymes is performed at a temperature that does not denature said one or more enzymes. Each step and method of the processes described herein which comprise the use of one or more enzymes can be performed at a pH in a range from about 3 to 10 or from about 4 to 10.

A “nucleobase” (or“nitrogenous base” or “base”) is a pyrimidine or purine heterocycle of nucleic acids such as DNA and RNA. As used herein, nucleobase includes adenine, guanine, cytosine, thymine or uracil, as well as nucleobases with non-natural modifications, for example, wherein the base has one or more non-natural substituents, or a modification affecting heteroatom(s) in a base excluding any change to the anomeric C-N linkage.

A 4′-ethynyl-2′-deoxy nucleoside contains a nucleobase. As used herein, an analog of a 4′-ethynyl-2′-deoxy nucleoside means a non-natural modification to the base of the nucleoside, for example wherein the base has one or more non-natural substituents, or a modification affecting heteroatom(s) in the base excluding any change to the anomeric C-N linkage.

As used herein, “phosphopentomutase” (“PPM”) enzymes (e.g. EC 5.4.2.7) arc enzymes that catalyze the reversible isomerization of ribose 1-phosphate to ribose 5-phosphate and related compounds such as deoxyribose phosphate and analogs of ribose phosphate and deoxyribose phosphate.

As used herein, “purine nucleoside phosphorylase” (“PNP”) enzymes (EC 2.4.2.2) are enzymes that catalyze the reversible phosphorolysis of purine ribonucleosides and related compounds (e.g., deoxyribonucleosides and analogs of ribonucleosides and deoxyribonucleosides) to the free purine base and ribose-1-phosphate (and analogs thereof).

As used herein, “sucrose phosphorylase” (“SP”) enzymes (EC 2.4.1.7) are enzymes that catalyze the reversible phosphorolysis of sucrose to D-fructose base and glucose-1-phosphate (and analogs thereof). Sucrose phosphorylase (SP) in combination with sucrose is employed in combination with purine nucleoside phosphorylase (PNP) and phosphomutase (PPM) to remove free phosphate ions from the reaction, where the combination of the enzymes catalyzes the formation of nucleoside MK-8591 (EFdA), while in some embodiments it could be replaced by other methods known in the art.

As used herein, “deoxyribose-phosphate aldolase” (“DERA”) (e.g., EC 4.1.2.4) refers to an enzyme in a family of lyases that reversibly cleave or create carbon-carbon bonds. Deoxyribose-phosphate aldolases as used herein include naturally occurring (wild type) deoxyribose-phosphate aldolase as well as non-naturally occurring engineered polypeptides generated by human manipulation. The wild-type deoxyribose-phosphate aldolase catalyzes the reversible reaction of 2-deoxy-D-ribose 5-phosphate into D-glyceraldehyde 3-phosphate and acetaldehyde.

As used herein, “pantothenate kinase,” (“PanK”) refers to enzymes (EC 2.7.1.33) which in nature phosphorylate pantothenate to form 4′-phosphopantothenate. Variant enzymes derived from such PanK enzymes may display improved activity and stereoselectivity towards 3′OH-group of D-ethynylglyceraldehyde regardless of whether such variants retain their natural function towards pantothenate.

As used herein, “galactose oxidase” (“GOase”; EC 1.1.3.9) enzymes are copper-dependent enzymes, that, in the presence of bimolecular oxygen, catalyze the oxidation of primary alcohols to the corresponding aldehydes. They act in both regio-and enantiospecific manners, enabling synthetic approaches that require little or no functional group protection and yield the desired stereoisomer. The manner of oxidation is mild and controlled, such that activity does not lead to over-oxidation of the alcohol to its corresponding carboxylic acid.

As used herein, “horseradish peroxidase” (HRP, EC 1.11.1.7) enzyme is an iron-dependent enzyme that activates and maintains GOase catalytic activity by oxidizing an inactive redox state of the active site that occurs during normal GOase catalytic cycling. Type I HRP is employed in a catalytic manner in the examples included herein, however it is not meant to be exclusive in this role, as there are other electron-transferring enzymes that belong to this and other enzyme classes as well as chemical reagents that can fulfill this role.

As used herein, “catalase” refers to a heme-dependent enzyme (EC 1.11.1.6) which acts on hydrogen peroxide, a byproduct of galactose oxidase or pyruvate oxidase reactions, which can render the enzymes inactive above certain levels of hydrogen peroxide. Catalase is employed as a catalytic maintenance enzyme in the examples herein to convert hydrogen peroxide to water and oxygen, while in some embodiments it could be replaced by other methods, such as electrochemical decomposition of hydrogen peroxide. A heme-dependent catalase is employed in a catalytic manner in the examples included herein, however it is not meant to be exclusive in this role, as there are other enzymes that belong to this class that can fulfill this role.

As used herein, “acetate kinase” (“AcK”) refers to an enzyme (EC 2.7.2.1), which catalyzes the formation of acetyl phosphate from acetate and adenosine triphosphate (ATP). It can also catalyze the reverse reaction, where it phosphorylates adenosine 5′-diphosphate (ADP) to adenosine 5′-triphosphate (ATP) in the presence of acetyl phosphate. Acetate kinase is employed to recycle ATP required by pantothenate kinase (PanK) in the examples herein, while in some embodiments the acetyl phosphate-acetate kinase recycling combination could be replaced by other methods known in the art.

As used herein, “pyruvate oxidase” (“PO”) refers to an enzyme (EC 1.2.3.3) dependent on Flavin adenine dinucleotide (FAD) and Thiamin diphosphate. Pyruvate oxidase is an enzyme belonging to the family of oxidoreductases, specifically those acting on the aldehyde or oxo group of a donor with oxygen as acceptor and it catalyzes the chemical reaction between pyruvate, phosphate ion and bimolecular oxygen to form acetyl phosphate, carbon dioxide and hydrogen peroxide. Pyruvate oxidase (PO) is employed in combination with acetate kinase (AcK) and catalase as a catalytic ATP-regenerating combination in the examples herein, where the combination of the enzymes catalyzes the formation of ATP from ADP in the presence of oxygen, pyruvate and phosphate ions, while in some embodiments it could be replaced by other methods known in the art.

As used herein, “wild-type” and “naturally-occurring” enzyme refers to the form found in nature. For example, a wild-type polypeptide sequence is a sequence present in an organism that can be isolated from a source in nature and which has not been intentionally modified by human manipulation.

As used herein, “engineered,” “variant,” “mutant” and “non-naturally occurring” when used with reference to an enzyme including a polypeptide, refers to a material, or a material corresponding to the natural or native form of the material, that has been modified in a manner that would not otherwise exist in nature. In some embodiments, the polypeptide is identical to a naturally occurring polypeptide, but is produced or derived from synthetic materials and/or by manipulation using recombinant techniques.

“Percentage of sequence identity,” “percent identity,” and “percent identical” with respect to enzymes are used herein to refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see e.g., Altschul et al., 1990, J. Mol. Biol. 215: 403-410 and Altschul et al., 1977, Nucleic Acids Res. 3389-3402). Software for performing BLAST analyses is publicly available through the National Center for Biotechnology Information website.

Briefly, the BLAST analyses involve first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation € of 10, M=5, N =-4, and a comparison of both strands. For amino acid sequences, the BLASTP program uses as defaults a wordlength (W) of 3, an expectation €(E) of 10, and the BLOSUM62 scoring matrix (see Henikoff and Henikoff, 1989, Proc Natl Acad Sci USA 89:10915).

Numerous other algorithms are available that function similarly to BLAST in providing percent identity for two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., cds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelerys, Madison WI), using default parameters provided.

“Substantial identity” refers to a polynucleotide or polypeptide sequence that has at least 85%, 86%, 87%, 88%, 89%, 90%, 91%, 92%, 93%, 94%, 95%, 96%, 97%, 98%, 99%, or more sequence identity, as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, more preferably at least 95 percent sequence identity or more (e.g., 99 percent sequence identity). Preferably, residue positions which are not identical differ by conservative amino acid substitutions.

“Stereoselectivity” refers to the preferential formation in a chemical or enzymatic reaction of one stereoisomer over another. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. It is commonly alternatively reported in the art (typically as a percentage) as the enantiomeric excess (e.e.) calculated therefrom according to the formula [major enantiomer−minor enantiomer]/[major enantiomer+minor enantiomer]. Where the stereoisomers are diastereoisomers, the stereoselectivity is referred to as diastereoselectivity, the fraction (typically reported as a percentage) of one diastereomer in a mixture of two diastereomers, commonly alternatively reported as the diastereomeric excess (d.e.). Enantiomeric excess and diastereomeric excess are types of stereomeric excess.

The phrase “suitable reaction conditions” refers to those conditions in the enzymatic conversion reaction solution (e.g., ranges of enzyme loading, substrate loading, temperature, pH, buffers, co-solvents, etc.) under which each polypeptide used in the present invention is capable of converting a substrate to the desired product compound. Some exemplary suitable reaction conditions are provided herein.

As used herein, “substrate” in the context of an enzymatic conversion reaction process refers to the compound or molecule acted on by the engineered enzymes used herein.

As used herein, “product” in the context of an enzymatic conversion process refers to the compound or molecule resulting from the action of an enzymatic polypeptide on a substrate.

As used herein, “increasing” yield of a product (e.g., a 4′ -ethynyl-2′-deoxyribose phosphate analog or 4′-ethynyl-2′-deoxy nucleoside analog) from a reaction occurs when a particular component present during the reaction (e.g., an enzyme) causes more product to be produced, compared with a reaction conducted under the same conditions with the same substrate but in the absence of the component of interest.

As used herein, “equilibration” or “equilibrium” as used herein refers to the process resulting in a steady state concentration of chemical species in a chemical or enzymatic reaction (e.g., interconversion of two species A and B), including interconversion of stereoisomers, as determined by the forward rate constant and the reverse rate constant of the chemical or enzymatic reaction.

“Enantiomeric excess” (ee) is a measurement of purity used for chiral substances. It reflects the degree to which a sample contains one enantiomer in greater amounts than the other. For example, a racemic mixture has an e.e. of 0%, while a single completely pure enantiomer has an e.e. of 100%; and a sample with 70% of one enantiomer and 30% of the other has an c.c. of 40% (70%−30%). Diastereomer excess (de) is calculated the same way as e.e. when only two diastereoisomers are present in the mixture.

“Protein”, “enzyme,” “polypeptide,” and “peptide” are used interchangeably herein to denote a polymer of at least two amino acids covalently linked by an amide bond, regardless of length or post-translational modification (e.g., glycosylation, phosphorylation, lipidation, myristilation, 10 ubiquitination, etc.). Included within this definition are D-and L-amino acids, and mixtures of D-and L-amino acids.

As used herein, the term “about” means an acceptable error for a particular value. In some instances “about” means within 0.05%, 0.5%, 1.0%, or 2.0% at the lower end and the upper end of given value range. With respect to pH, “about” means plus or minus 0.5.

As used herein, “substantially pure” polypeptide or “purified” protein refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. However, in some embodiments, the composition comprising the polypeptide comprises polypeptide that is less than 50% pure (e.g., about 10%, about 20%, about 30%, about 40%, or about 50%). Generally, a substantially pure polypeptide composition comprises about 60% or more, about 70% or more, about 80% or more, about 90% or more, about 95% or more, and about 98% or more of all macromolecular species by mole or % weight present in the composition. In some embodiments, the polypeptide is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated polypeptides are substantially pure polypeptide compositions.

As used herein, “improved property” of an enzyme refers to at least one improved property of an enzyme. In some embodiments, the present invention employs engineered PPM, PNP, DERA, PanK, AcK, SP and/or GOase polypeptides that exhibit an improvement in any enzyme property as compared to a reference PPM, PNP, DERA, PanK, AcK, SP or GOase polypeptide, respectively, and/or a wild-type PPM, PNP, DERA, PanK, AcK, SP or GOasc polypeptide, respectively, and/or another engineered PPM, PNP, DERA, PanK, AcK, SP or GOase polypeptide, respectively. Thus, the level of “improvement” can be determined and compared between the various polypeptides, including wild-type, as well as engineered polypeptides. Improved properties include, but are not limited, to such properties as increased protein expression, increased production of the intended product, increased substrate specificity or affinity (i.e., increased activity on the substrate), increased thermoactivity, increased thermostability, increased pH activity, increased stability, increased enzymatic activity, increased specific activity, increased resistance to substrate or end-product inhibition, increased chemical stability, improved chemoselectivity, improved solvent stability, increased tolerance to acidic pH, increased tolerance to proteolytic activity (i.e., reduced sensitivity to proteolysis), reduced aggregation, increased solubility, and altered temperature profile. In additional embodiments, the term is used in reference to the at least one improved property of PPM, PNP, DERA, PanK, AcK, SP and/or GOase enzymes. In some embodiments, the present invention employs engineered PPM, PNP, DERA, PanK, AcK, SP and/or GOase polypeptides that exhibit an improvement in any enzyme property as compared to a reference PPM, PNP, DERA, PanK, AcK, SP and/or GOase polypeptide, respectively; and/or a wild-type polypeptide, and/or another engineered PPM, PNP, DERA, PanK, AcK, SP and/or GOase polypeptide, respectively. Thus, the level of “improvement” can be determined and compared between the various polypeptides, including wild-type, as well as engineered polypeptides.

As used herein, “conversion” (“conv” or “conv.”) refers to the enzymatic conversion (or biotransformation) of a substrate(s) to the corresponding product(s). “Percent” conversion refers to the percent of the substrate that is converted to the product within a period of time under specified conditions. Thus, the “enzymatic activity” or “activity” of a polypeptide can be expressed as percent conversion of the substrate to the product in a specific period of time.

As used herein, “stereoselectivity” refers to the preferential formation in a chemical or enzymatic reaction of one stereoisomer over another. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. It is commonly alternatively reported in the art (typically as a percentage) as the enantiomeric excess (“e.e.”) calculated therefrom according to the formula [major enantiomer−minor enantiomer]/[major enantiomer+minor enantiomer]. Where the stereoisomers are diastereoisomers, the stereoselectivity is referred to as diastereoselectivity, the fraction (typically reported as a percentage) of one diastereomer in a mixture of two diastereomers, commonly alternatively reported as the diastereomeric excess (“d.e.”). Enantiomeric excess and diastereomeric excess are types of stereomeric excess.

The present process invention encompasses the use of engineered PPM, PNP, DERA, PanK, AcK, SP and GOase polypeptides, particularly those having SEQ ID NO.s 1 to 21, and said sequences which comprise one or more conservative amino acid substitutions which may be referred to as conservatively modified variants of each of SEQ ID NO.s 1 to 21.

As used herein, “conservative” amino acid substitution and refers to substitutions of amino acids in a protein with other amino acids having similar characteristics (e.g. acidic, basic, positively or negatively charged, polar or non-polar, side-chain size, hydrophobicity/hydrophilicity, backbone conformation and rigidity, etc.), such that the changes can frequently be made without altering the biological activity of the protein. This includes one or more substitutions of an amino acid in the polypeptide with a different amino acid within the same or similar defined class of amino acids. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. By way of example and not limitation, in some embodiments, an amino acid with an aliphatic side chain is substituted with another aliphatic amino acid (e.g., alanine, valine, leucine, and isoleucine); an amino acid with an hydroxyl side chain is substituted with another amino acid with an hydroxyl side chain (e.g., serine and threonine); an amino acid having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively. Additional exemplary conservative amino acid substitutions are set forth in Table 1.

TABLE 1
Exemplary Conservative Amino Acid Substitutions

| Original | Conservative |
|---|---|
| residue | substitution |
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

The term “amino acid substitution set” or “substitution set” refers to a group of amino acid substitutions in a polypeptide sequence, as compared to a reference sequence. A substitution set can have 1, 2, 3, 4, 5, 6, 7, 8, 9, 10, 11, 12, 13, 14, 15, or more amino acid substitutions.

A “functional fragment” refers to a polypeptide that has an amino-terminal and/or carboxy-terminal deletion(s) and/or internal deletions, but where the remaining amino acid sequence is identical to the corresponding positions in the sequence to which it is being compared (e.g., a full-length engineered PPM, PNP, DERA, PanK, AcK, SP or GOase enzyme used in the present invention) and that retains substantially all of the activity of the full-length polypeptide.

As used herein, “deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered PPM, PNP, DERA, PanK, AcK, SP or GOase enzyme. Deletions can be directed to the internal portions and/or terminal portions of the polypeptide. In various embodiments, the deletion can comprise a continuous segment or can be discontinuous. Deletions are typically indicated by “-” in amino acid sequences.

As used herein, “insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

Additional acronyms and abbreviations used herein are as follows:

| | |
|---|---|
| LC-MS | liquid chromatography mass spectrometry |
| THF | tetrahydrofuran |
| NMR | nuclear magnetic resonance spectroscopy |
| RT or rt | room temperature (ambient, about 25° C.) |
| sccm | standard cubic centimeter per minute |
| rpm | revolutions per minute |
| M | mole/molarity |
| mM | millimolar |
| μL | microliter(s) |
| DMSO | dimethyl sulfoxide |
| TsOH | p-toluenesulfonic acid |
| Bn | benzyl |
| CPME | Cyclopentyl methyl ether |
| MTBE | methyl tert-butyl ether |
| HR-MS | High Resolution Mass Spectrometry |
| g/L | gram(s) per liter |
| mL | milliliter(s) |
| mmol | millimole |
| mg | milligram |
| kg | kilogram |
| N | Normal |
| conv | conversion |
| NMR | nuclear magnetic resonance |
| aq | aqueous |
| hr, h | hour(s) |
| HPLC | high performance liquid chromatography |
| DCM | dichloromethane |
| 2-MeTHF | 2-Methyltetrahydrofuran |
| ESI | Electrospray ionization |

### Experimental Procedures

Preparation of 2-ethynyl-2-hydroxypropane-1,3-diyl Diacetate (2)

### Method A:

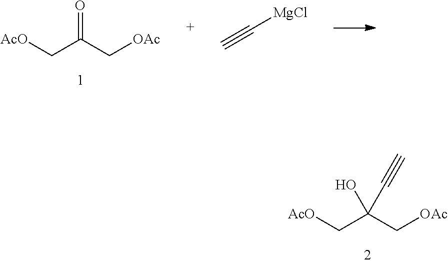

To a −35° C. solution of diacetoxyacetone (1) (159 g, 914.0 mmol) in THF (1000 mL) was added 1600 mL of a 0.5 M solution of ethynyl magnesium chloride in THF maintaining the temperature below −20° C. After the reaction reached completion, acetic acid (78 mL) in 400 mL methyl tert-butyl ether (MTBE) was added dropwise keeping the temperature below −20° C. MTBE (800 mL) was then added and the mixture was warmed to room temp. Saturated NaCl in water (1000 mL) was added followed by saturated NH₄Cl solution in water (1050 mL). The organic layer was separated, dried over Na₂SO₄ and evaporated to give compound (2) as an oil (160 g, 88%). ¹H NMR (CDCl₃, 500 MHz): δ 4.26 (dd, 4 H), 2.55 (s, 1H), 2.14 (s, 6H).

Preparation of 2-ethynyl-propane-1,2,3-triol (3)

### Method B:

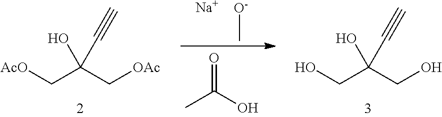

To a solution of 2-ethynyl-2-hydroxypropane-1,3-diyl diacetate (2) (70 g, 350 mmol) in ethanol was added a 0.5M solution of sodium methoxylate in methanol (69.9 mL, 35.0 mmol) at room temperature (rt). The reaction was stirred at rt for 2 hours (h) to reach completion. The solvents were evaporated and the residue was re-dissolved in 100 mL water and extracted with 3×50 mL MTBE. The aqueous layer was sparged with nitrogen to remove residual solvents to give a 40.9% solution of 2-ethynyl-propane-1,2,3-triol (3) (108 g , 100% yield) as determined by nuclear magnetic resonance (NMR) (maleic acid as internal standard). ¹H NMR (D₂O, 500 MHz): δ 3.60 (dd, 4 H), 2.85 (s, 1H).

Alternate Preparations of (R)-2-ethynyl-glyceraldehyde (4)

### Method C1:

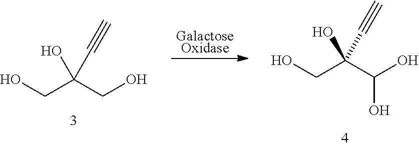

In a stirred reactor, 2-ethynyl-propane-1,2,3-triol (3) (1.1 g, 9.47 mmol) in sodium phosphate buffer (30 mL, 100 mM, pH 7.0) containing antifoam 204 (Sigma A6426, 1 drop˜20 μL) was warmed to 30° C. with air sparging at 12.5 sccm. Galactose oxidase (GOase, SEQ ID NO.: 1) (250 mg), Horseradish Peroxidase* (Type I, 5 mg) and bovine catalase ** (5 mg) dissolved in sodium phosphate buffer (5 mL 100 mM, pH 7.0) were added to the reactor, followed by the addition of CuSO₄ aq. solution (100 mM, 150 μL). The reaction mixture was stirred at 600 rpm with air sparging for 47 h to give (R)-2-ethynyl-glyceraldehyde (4) in 47% conversion (by NMR) and 72% e.e. . (The product was not isolated). ¹H NMR (D₂O, 500 MHz): δ 4.29 (s, 1H), 3.65 (dd, 2H), 2.83 (s, 1H).

* Horse Radish Peroxidase: wild type peroxidase from horseradish Type I, commercially available from SIGMA (P8125), isolated from horseradish roots (Amoracia rusticana).
** Bovine catalase: heme-dependent catalase from bovine source, commercially available from Sigma (C1345)

Method C2:

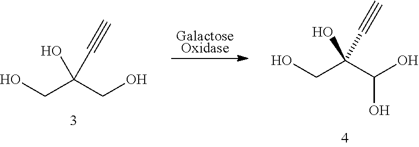

In a stirred 100 L jacketed reactor charged with deionized water (56.2 kg), sodium dihydrogen phosphate (1.212 kg, 10 moles) was added. The pH was adjusted to 7.02 using 10 N sodium hydroxide solution (852.6 g) at 25° C. The reactor was charged with Antifoam 204 (A6426, 10 mL), followed CuSO₄·5H₂O (6.5 g). Galactose oxidase (451.2 g) (SEQ ID NO.: 10) was added and stirred for 15 min while sparged with air. Horseradish peroxidase* (200.2 g) and catalase ** (502.6 g) were added and the reactor was rinsed with water (2.0 kg). Next 2-ethynyl-propane-1,2,3-triol (3) solution in water (9.48%, 30.34 kg, 24.72 mol) was added followed by an additional portion of Antifoam 204 (A6426, 10 mL). The reaction was sparged with air and stirred overnight to give 94.0 kg of (R)-2-ethynyl-glyceraldehyde (4) in 66% conversion (by NMR) and 84% e.e. Assay yield 60%: ¹H NMR (D₂O, 500 MHZ): δ 4.29 (s, 1H), 3.65 (dd, 2H), 2.83 (s, 1H).

* Horse Radish Peroxidase: wild type peroxidase from horseradish purified, commercially available from Toyobo (PEO-301), isolated from horseradish roots (Amoracia rusticana).
** Bovine catalase: heme-dependent catalase from bovine source, commercially available from Sigma (C1345).

The above reaction was also performed using the galactose oxidase (SEQ ID NO.: 11) and the product (4) was obtained in 67% conversion (by NMR) and 88% e.e. and assay yield 59%: ¹H NMR (D₂O, 500 MHz): δ 4.29 (s, 1H), 3.65 (dd, 2H), 2.83 (s, 1H).

Method C3:

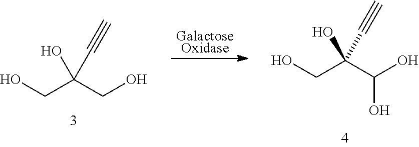

In a 100 mL EasyMax vessel equipped with sparger and flow controller, water (82 mL) and PIPES potassium buffer (5 mL, 0.5 M) were charged. The pH was adjusted to 7.5 using 5 M KOH solution at 25° C. Antifoam 204 (200 μL) was added, followed by evolved galactose oxidase (SEQ ID NO.: 17, 450 mg enzyme powder) and copper(II) sulfate pentahydrate (100 μL, 100 mM). The reaction mixture was sparged with air at 125 standard cubic centimeters per minute (sccm) for 15 min. Bovine catalase (C1345, Sigma-Aldrich, 150 mg, 2000-5000 U/mg, 0.75 MU) was charged, followed by horseradish peroxidase (HRP, Toyobo PEO-301, 100 mg, 130 U/mg, 1.3 kU) and the aqueous solution of 2-ethynyl-propane-1,2,3-triol (3) (25 wt %, 12 mL, 25.8 mmol). The reaction mixture was stirred at 30° C. with aeration at 125 sccm and sampled using EasySampler over 20 h to give 70% conversion and form compound (4) ((R)-2-ethynyl-glyceraldehyde) in 58% assay yield and 99% e.e. ¹H NMR (D₂O, 500 MHz): δ 4.29 (s, 1H), 3.65 (dd, 2H), 2.83 (s, 1H). The crude reaction stream was carried directly into the subsequent phosphorylation step.

Method C4: Oxidation with Immobilized Galactose Oxidase

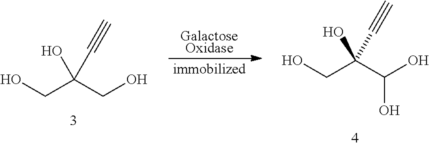

### Enzyme Immobilization Procedure:

Nuvia IMAC Ni-charged resin (16 mL based on settled volume) was added to a filter funnel and washed with binding buffer (10 column volumes, 160 mL; 500 mM sodium chloride, 50 mM sodium phosphate, 15 mM imidazole, pH 8.0) to remove the resin storage solution. In a vessel evolved galactose oxidase (SEQ ID NO.: 17, 2.00 g) lyophilized powders were resuspended in copper (II) sulphate solution (100 μM; 5.00 mL), followed by addition of binding buffer (50 mL) and the resin. The solution was mixed using rotating mixer at 20° C. for 5 h. The resin was filtered and washed with binding buffer (10 column volumes, 160 mL) and potassium PIPES buffer (10 column volumes, 160 mL; 50 mM, pH 7.5) and it was used directly in a reaction.

### Reaction Procedure:

In a 100 mL EasyMax vessel equipped with sparger and flow controller, water (82 mL) and PIPES potassium buffer (5 mL, 1 M) were charged. The pH was adjusted to 7.5 using 5 M KOH solution at 25° C. Antifoam 204 (200 μL) was added, followed by evolved galactose oxidase immobilized on the resin (SEQ ID NO.: 17, 750 mg enzyme powder per 6 mL resin) and copper(II) sulfate pentahydrate (100 μL, 100 mM). The reaction mixture was sparged with air at 125 standard cubic centimeters per minute (sccm) for 15 min. Bovine catalase (C1345, Sigma-Aldrich, 210 mg, 2000-5000 U/mg, 1.05 MU) was charged, followed by horseradish peroxidase (HRP, Toyobo PEO-301, 100 mg, 130 U/mg, 1.3 kU) and the aqueous solution of 2-ethynyl-propane-1,2,3-triol (3) (25 wt %, 13 mL, 29.4 mmol). The reaction mixture was stirred at 25° C. with aeration at 125 sccm. After 22 h the reaction reached 91% conversion to give 200 mM (R)-2-ethynyl-glyceraldehyde (4) solution (100 mL, 68% assay yield, 97% e.e. ¹H NMR (D₂O, 500 MHz): δ 4.29 (s, 1H), 3.65 (dd, 2H), 2.83 (s, 1H). The crude reaction stream was carried directly into the subsequent phosphorylation step.

### Method C5: Optional Isolation of Aldehyde via Formation of Aminal (8)

Step 1: Preparation of (S)-2-(1,3-dibenzylimidazolidin-2-yl)but-3-yne-1,2-diol

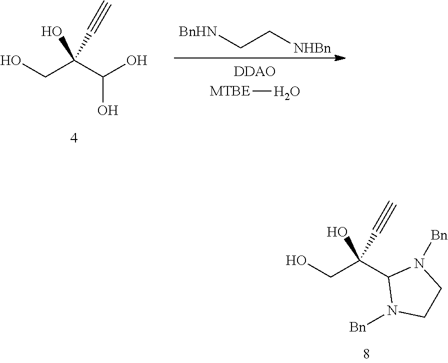

A 100 L jacketed cylindrical vessel equipped with nitrogen bubbler, mechanical stirrer and thermocouple was charged with crude oxidase reaction stream containing (R)-2-ethynyl-glyceraldehyde ((4), 26.0 kg, 1.85 wt % aldehyde, 3.64 mol) and inerted with N₂ atmosphere. The aqueous solution was warmed to 20° C. and N,N-dimethyldodecan-1-amine oxide (DDAO) (30 wt % in water, 798 g, 0.96 mol;) was added, followed by MTBE (55.3 kg, 76 L) and N,N′-dibenzylethane-1,2-diamine (1.55 kg, 6.43 mol). The brown, biphasic mixture was stirred overnight at 20° C. under nitrogen atmosphere. After 17 hours the stirring was stopped and the organic phase was removed and discarded. A light brown MTBE solution of (S)-2-(1,3-dibenzylimidazolidin-2-yl)but-3-yne-1,2-diol (56.5 kg, 2.02 wt % aminal, 3.39 mmol, 93% assay yield) was obtained.

Six similar MTBE solutions were processed together in a single distillation and crystallization step (in total 374.4 kg of solution, containing 7.91 kg aminal).

A 50 L jacketed cylindrical vessel equipped with mechanical stirrer, distillation head (condenser at-20° C.) and thermocouple was charged with aminal solution (45 L). Vacuum was applied to the vessel (65-95 torr) and the jacket was set to 40° C. Solvent was removed by distillation until a volume of 35 L had been reached. At this point, the internal temperature was 6.1° C. and an off-white solid had begun to crystallize. The remaining MTBE solution was slowly added, maintaining a constant volume of 35-40 L and an internal temperature of 0-10° C. Once all the MTBE solution had been added the volume was decreased to 25 L. Distillation was halted, the vessel was inerted with nitrogen and the jacket temperature was decreased to 10° C. The resulting pale yellow suspension was aged at this temperature for 2 hours and the solids were collected by filtration. The filter cake was washed with cold (−2° C.) MTBE (12.7 kg) and then dried under nitrogen flow for 7 hours. (S)-2-(1,3-dibenzylimidazolidin-2-yl)-but-3-yne-1,2-diol was obtained as an off-white crystalline solid (5.75 kg). ¹H NMR (500 MHZ, DMSO-d6) 8 7.42-7.35 (m, 4H), 7.32 (td, J=7.5, 1.6 Hz, 4H), 7.27-7.21 (m, 2H), 5.10 (t, J=5.6 Hz, 1H), 5.03 (s, 1H), 4.28 (d, J=13.3 Hz, 1H), 4.16 (d, J=13.3 Hz, 1H), 3.76 (s, 1H), 3.70-3.58 (m, 4H), 3.21 (d, J=0.9 Hz, 1H), 2.90-2.80 (m, 2H), 2.60-2.51 (m, 2H). 13C NMR (126 MHZ, DMSO-d6) δ 140.0, 140.0, 128.5, 128.3, 128.2, 128.1, 126.8, 126.8, 88.6, 86.9, 75.0, 74.0, 66.4, 60.7, 60.5, 50.4, 50.3, 39.5. HR-MS (ESI) Aminal (M+H⁺) C₂₁H₂₅N₂O₂⁺ calculated 337.1911; found 337.1922.

Step 2: Preparation of (R)-2-ethynyl-glyceraldehyde (4) from Aminal (8)

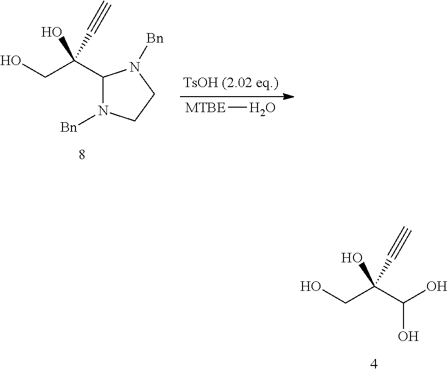

A 4 L jacketed cylindrical vessel equipped with nitrogen bubbler and mechanical stirrer was charged with of TsOH·H₂O (12.0 g, 63.1 mmol), water (60 mL), (S)-2-(1,3-dibenzylimidazolidin-2-yl)but-3-yne-1,2-diol (110 g, 327 mmol) and MTBE (1700 mL). The biphasic mixture was placed under nitrogen and the jacket temperature was set to 15° C. A solution of TsOH·H₂O (114 g, 599.3 mmol) in water (600 mL) was added dropwise over 1.5 hours with overhead stirring (200 rpm). After addition had completed, the jacket temperature was lowered to 5° C. and the resulting slurry was aged for 1 hour. The solids were removed by filtration and washed with cold water (270 mL). The biphasic solution was transferred to a separating funnel and the organic phase was removed and discarded. The aqueous phase was treated with DOWEX™ MARATHON™ A resin (hydroxide form, 11.0 g) and AMBERLYST® 15 resin (hydrogen form, 11.0 g) while sparging with N₂ at a rate of 200 sccm for 24 hours to remove residual MTBE. The resins were removed by filtration to give a colorless aqueous solution of (R)-2-hydroxy-2-(hydroxymethyl)but-3-ynal (774 g, 4.6 wt % aldehyde, 82% yield). ¹H NMR (500 MHZ, D₂O) δ 5.01 (s, 1H), 3.77 (d, J=11.7 Hz, 1H), 3.73 (d, J=11.7 Hz, 1H), 2.92 (s, 1H). ¹³C NMR (126 MHz, D₂O) δ 129.4, 125.4, 90.3, 81.0, 76.0, 73.9, 65.3. HRMS (ESI) Aldehyde dimer (2M+Na⁺) C₁₀H₁₂NaO₆⁺ calculated 251.0526; found 251.0530.

Alternate Preparations of (R)-2-ethynyl-glyceraldehyde 3-phosphate (5): Method D1: Acetate kinase: ATP-regeneration system

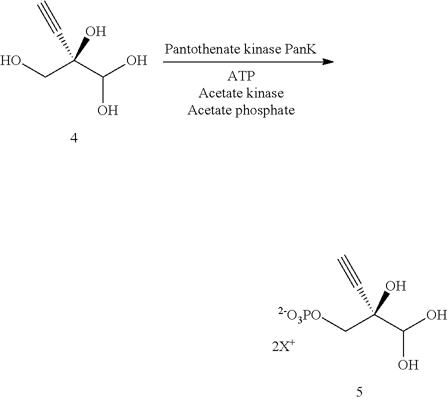

In a stirred reactor, to a solution of adenosine diphosphate disodium salt (40 mg, 0.087 mmol) and magnesium chloride (38 mg, 0.400 mmol) in HEPES buffer (66 mM, pH 7.5, 30 mL) was added (R)-2-ethynyl-glyceraldehyde (4) (1.9 mL, 210 g/L solution in water, 3.51 mmol), followed by acetate kinase (SEQ ID NO.: 3) (40 mg), and pantothenate kinase (SEQ ID NO.: 2) (120 mg). The reaction mixture was warmed to 25° C. and a solution of acetyl phosphate lithium potassium salt (1.3 g, 7.01 mmol) in HEPES buffer (50 mM, pH 7.5, 10 mL) was added dropwise over 4 hours, with pH maintained at 7.5 using 5M sodium hydroxide. The reaction was stirred for 18 hours to give (R)-2-ethynyl-glyceraldehyde 3-phosphate (5) in 85% conversion (by HPLC) (The product was not isolated). ¹H NMR (D₂O, 400 MHZ): δ 5.02 (s, 1H), 4.00 (dq, 2 H), 2.88 (s, 1H). LC-MS: (ES, m/z): calculated for C₅H₇O₆P (M⁻H): 193.1; found 193.0.

### Method D2: Pyruvate Oxidase ATP-regeneration System

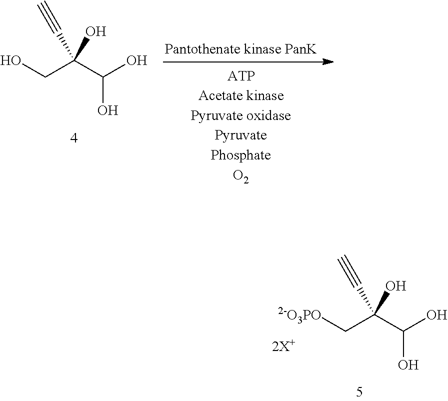

In a stirred reactor, a solution of sodium pyruvate (3.11 g, 28 mmol) and phosphoric acid (0.523 mL, 7.71 mmol) in 76 mL water pH 7.5 was charged with (R)-2-ethynyl-glyceraldehyde (4) (3.8 mL, 210 g/L solution in water, 7.01 mmol), adenosine diphosphate disodium salt (80 mg, 0.174 mmol), thiamine pyrophosphate (40 mg, 0.086 mmol), flavin adenine dinucleotide disodium salt hydrate (64 mg, 0.077 mmol), and magnesium chloride (400 μL, 1 M solution in water, 0.4 mmol). The pH was re-adjusted to 7.5 with 5M aq sodium hydroxide and the reaction volume was re-adjusted to 80 mL with water. Acetate kinase (SEQ ID NO.: 3) (80 mg), pyruvate oxidase (SEQ ID NO.: 4) (80 mg, lyophilized cell free extract), pantothenate kinase (SEQ ID NO.: 2) (400 mg), and catalase (800 μL, ammonium sulfate suspension CAT-101, Biocatalytics) were added. The reaction was stirred at 500 rpm and 30° C. with air sparging for 72 hours to give (R)-2-ethynyl-glyceraldehyde 3-phosphate 5 in 95% conversion (by HPLC) (The product was not isolated). ¹H NMR (D₂O, 400 MHZ): δ 5.02 (s, 1H), 4.00 (dq, 2 H), 2.88 (s, 1H). LC-MS: (ES, m/z): calculated for C₅H₇O₆P (M⁻H): 193.1; found 193.0.

The above reaction was also performed using the pantothenate kinase (SEQ ID NO.: 13) and the product 5 was obtained in 66% conversion. (The product was not isolated). ¹H NMR (D₂O, 400 MHZ): δ 5.02 (s, 1H), 4.00 (dq, 2 H), 2.88 (s, 1H).

### Method D3: Acetate Kinase: ATP-regeneration System Using Immobilized Enzymes

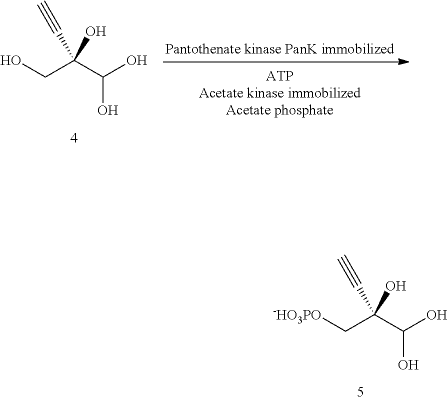

### Enzyme Immobilization Procedure:

NUVIA™ Immobilized Metal-ion Affinity Chromatography (IMAC) nickel-charged resin (168 mL based on settled volume) was added to a filter funnel and washed with binding buffer (1.6 L; 500 mM sodium chloride, 50 mM sodium phosphate, pH 8.0). In a vessel, pantothenate kinase (8.4 g) (SEQ ID NO.: 12) and acetate kinase (2.8 g) (SEQ ID NO.: 3) were dissolved in binding buffer (500 mL). The washed resin was charged to the vessel and the solution was stirred for 4 hours at 20° C. The resin was filtered and washed first with binding buffer (1.6 L) followed by piperazine-N,N′-bis(2-ethanesulfonic acid) (PIPES) buffer (840 mL; 50 mM, pH 6.5). The washed resin was used directly in the next step.

### Reaction Procedure:

To a 1 L reactor, a solution of (R)-2-ethynyl-glyceraldehyde (4) in water (608.7 g, 4.6 wt %, 212 mmol) was charged and cooled to 5° C. To the cooled solution piperazine-N,N′-bis(2-ethanesulfonic acid) (PIPES) buffer (32.7 mL, 1 M, pH 6.5, 32.7 mmol), magnesium chloride (9.33 mL, 1 M, 9.33 mmol), acetyl phosphate diammonium salt (51.8 g, 265 mmol), adenosine diphosphate disodium salt hydrate (1.17 g, 2.12 mmol), and water (192 mL) were added. The solution was allowed to stir and pH was adjusted to 6.4 using 5 N KOH. The reaction was warmed to 20° C. and 168 mL of resin with co-immobilized pantothenate kinase (SEQ ID NO.: 12) and acetate kinase (SEQ ID NO.: 3) was added. The reaction was stirred for 10 hours with 5 N KOH used to maintain a pH of 6.4 to give (R)-2-ethynyl-glyceraldehyde 3-phosphate (5) in 92% conversion (by HPLC) and 91% yield (by ³¹P NMR with tetraphenylphosphonium chloride as internal standard) (the product was not isolated). ¹H NMR (D₂O, 400 MHZ): δ 5.02 (s, 1H), 4.00 (dq, 2 H), 2.88 (s, 1H). LC-MS: (ES, m/z): calculated for C₅H₇O₆P (M⁻H): 193.1; found 193.0.

Preparation of 4-ethynyl-D-2-deoxyribose 5-phosphate (6)

### Method E:

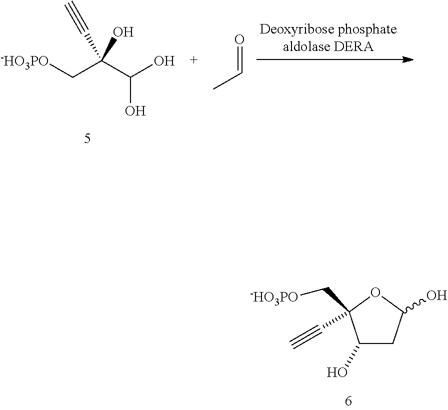

To a solution of (R)-2-ethynyl-glyceraldehyde 3-phosphate (5) (5, 20 mL, 5.3 mmol) in water, a solution of acetaldehyde in water (40 wt. %, 2.02 mL, 15.9 mmol) was added at room temperature, followed by the addition of Deoxyribose-phosphate aldolase (DERA) (SEQ ID NO.: 6), 25 mg solution in triethanolamine hydrochloride buffer (1 mL, 1 M, pH 7.0). The reactor was sealed and the mixture was stirred overnight at 30° C. and 600 rpm to give 4-ethynyl-D-2-deoxyribose 5-phosphate (6) in 99% conv. and 99% e.e., 99% d.e. as a 1:1 anomer mixture (The product was not isolated). α-anomer: ¹H NMR (D₂O, 600 MHz) δ 5.31 (t, 1H), 4.13 (t, 1H), 3.81-3.72 (m, 2H), 2.89 (s, 1H), 2.42-2.34 (m, 1H), 1.87-1.79 (m, 1H); ¹³C NMR (D₂O, 151 MHZ) δ 97.7 (s), 81.4 (d), 79.4 (s), 78.9 (s), 71.1 (s), 67.7 (d), 39.6 (s). B-anomer: ¹H NMR (D₂O, 600 MHz) δ 5.40 (dd, 1H), 4.28 (t, 1H), 3.88-3.80 (m, 2H), 2.87 (s, 1H), 2.13-2.06 (m, 1H), 2.04-1.97 (m, 1H); ¹³C NMR (D₂O, 151 MHZ) δ 97.3 (s), 82.2 (d), 78.7 (s), 78.5 (s), 71.3 (s), 68.4 (d), 39.6 (s). LC-MS: (ES, m/z): calculated for C₇H₁₀O₇P (M⁻H): 237.0; found 237.0

Alternate Preparations of (2R,3S,5R)-5-(6-amino-2-fluoro-9H-purin-9-yl)-2-ethynyl-2-(hydroxymethyl)tetrahydrofuran-3-ol monohydrate (7) [alternative name 4′-ethynyl-2-fluoro-2′-deoxyadenosine or EFdA]

### Method F1:

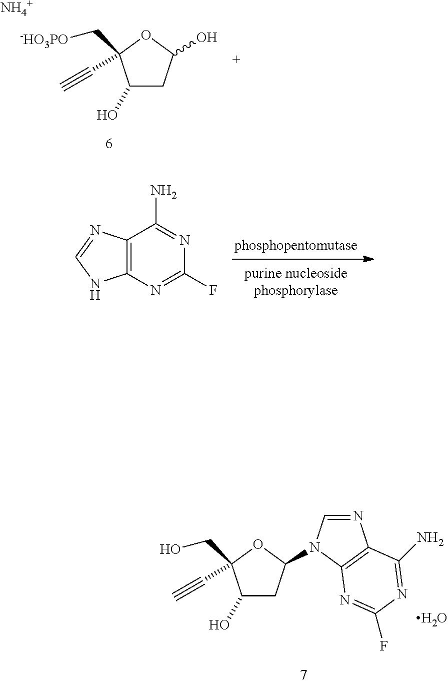

Ammonium ((2R,3S)-2-ethynyl-3,5-dihydroxytetrahydrofuran-2-yl)methyl hydrogen phosphate (1.00 g, 3.91 mmol) was dissolved in 10 mL of pH 7.5 buffer (100 mM triethanolamine. HCl containing 5 mM MnCl₂). The solution pH was adjusted to 7.3 with 5 N NaOH. To the solution was added 2-fluoroadenine (0.599 g, 3.91 mmol) and sucrose (2.68 g, 7.82 mmol). The enzyme solution was prepared by dissolving phosphopentomutase (SEQ ID NO.: 8) (100 mg), purine nucleoside phosphorylase (SEQ ID NO.: 9) (50 mg), and sucrose phosphorylase (SEQ ID NO.: 7) (10 mg) in 10 mL of the pH 7.5 buffer. The enzyme solution was added to the reagent mixture and the resulting suspension was shaken at 40° C. After 20 h, the suspension was cooled to 0° C. and filtered, rinsing with cold water. The solid was suction dried to give the title compound (1.12 g, 92%) as a single isomer.

¹H NMR: (300 MHz, DMSO-d6, ppm): δ 7.68 (br s, 2H), 7.32 (d, J=2.0 Hz, 1H), 6.44 (t, J =5.8 Hz, 1H), 5.52 (d, J=5.6 Hz, 1H), 5.27 (t, J=6.0 Hz, 1H), 4.44 (q, J=6.4 Hz, 1H), 3.60 (q, J =6.0 Hz, 1H), 3.53 (q, J=6.4 Hz, 1H), 3.48 (s, 1H), 2.48-2.41 (m, 1H), 2.37-2.30 (m, 1H). ¹³C NMR (150.92 MHz, DMSO-d6, ppm) δ 158.5 (d, JCF=203.5), 157.6 (d, JCF=21.2), 150.2 (d, JCF=20.2), 139.7 (d, JCF=2.4), 117.4 (d, JCF=4.0), 85.1, 82.0, 81.4, 78.7, 70.1, 64.2, 38.1. LC-MS: (ES, m/z): calculated for C₁₂H₁₂FN₅O₃ (M+Na): 316.0822; found 316.0818.

The PPM and PNP enzymes used in this step were each derived from mutations starting from the enzymes from E. coli (Escherichia coli). The sucrose phosphorylase (SP) used in this step was derived from Alloscardovia omnicolens; SP derived from other organisms could also be used.

### Method F2:

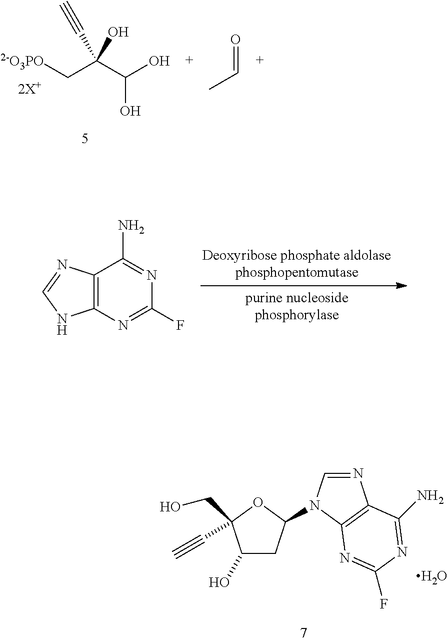

To an aqueous solution of (R)-2-ethynyl-glyceraldehyde 3-phosphate (5) (950 mL, 157 mmol) containing piperazine-N,N′-bis(2-ethanesulfonic acid) (PIPES) buffer at a pH from about 5.5 to 6.0 was added triethanolamine (7.09 g, 47.5 mmol). The pH of the solution was adjusted from 7.1 to 7.6 using potassium hydroxide (8 mL, 8M). Manganese(II) chloride hydrate (0.592 g, 4.70 mmol) was added followed by sucrose (161 g, 470 mmol), giving a pH of 7.5 To the solution was added the following enzymes: deoxyribose-phosphate aldolase (SEQ ID NO.: 14) (461 mg), sucrose phosphorylase (SEQ ID NO.: 7) (494 mg), phosphopentomutase (SEQ ID NO.: 8)(2.63 g), and purine nucleoside phosphorylase (SEQ ID NO.: 15) (659 mg). Once the enzymes were dissolved, 2-fluoroadenine (19.80 g, 125 mmol) was added. The reaction was heated to 35° C. and acetaldehyde was added (40 wt % in isopropyl alcohol, 29.8 mL, 235 mmol). After reacting for 2 h, the mixture was seeded with EFdA crystalline product (0.96 g, 2 mol %). After reacting over 26 h at 35° C., the slurry was cooled to 0° C., and the solids were collected by filtration, washing with water two times (40 mL ea.). The solids were dried under a nitrogen sweep. Yield 43.2 g, 92 wt %, 96.2% corrected. ¹H NMR: (300 MHz, DMSO-d6, ppm): δ 7.68 (br s, 2H), 7.32 (d, J=2.0 Hz, 1H), 6.44 (t, J=5.8 Hz, 1H), 5.52 (d, J=5.6 Hz, 1H), 5.27 (t, J =6.0 Hz, 1H), 4.44 (q, J=6.4 Hz, 1H), 3.60 (q, J=6.0 Hz, 1H), 3.53 (q, J=6.4 Hz, 1H), 3.48 (s, 1H), 2.48-2.41 (m, 1H), 2.37-2.30 (m, 1H). ¹³C NMR (150.92 MHz, DMSO-d6, ppm) § 158.5 (d, JCF=203.5), 157.6 (d, JCF=21.2), 150.2 (d, JCF=20.2), 139.7 (d, JCF=2.4), 117.4 (d, JCF=4.0), 85.1, 82.0, 81.4, 78.7, 70.1, 64.2, 38.1. LC-MS: (ES, m/z): calculated for C₁₂H₁₂FN₅O₃ (M+Na): 316.0822; found 316.0818.

Alternate Preparations of (S)-2-ethynyl-propane-1,2,3-triol 1 1-phosphate (9):

### Method G1: Acetate Kinase: ATP-regeneration System Using Enzymes SEQ. ID No.: 2 and SEQ. ID No.: 3

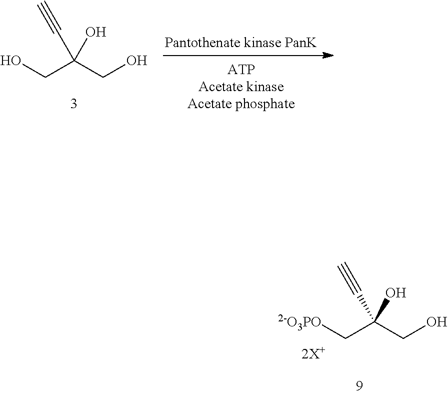

A 50 mL reactor was charged with a solution of 2-ethynyl-propane-1,2,3-triol (3) in water (9.29 g, 9.46 wt %, 7.57 mmol) potassium PIPES buffer (1.02 mL, 1 M, pH 6.5, 1.02 mmol), magnesium chloride (292 μL, 1 M, 0.292 mmol), acetyl phosphate diammonium salt (1.851 g, 89 wt %, 9.46 mmol), adenosine diphosphate disodium salt hydrate (ADP, 42 mg, 0.076 mmol, 0.01 eq), and water (28 mL). The pH was adjusted to 6.4 using 5 M KOH, the solution was warmed to 20° C. and evolved pantothenate kinase PanK SEQ. ID No.: 2 (264 mg) and acetate kinase AcK SEQ. ID No.: 3 (88 mg) were added. The reaction was stirred for 16 hours with pH maintained at 6.4 using 5 N KOH. The final reaction contents provided (S)-2-ethynyl-propane-1,2,3-triol 1-phosphate (9) in >95% e.e. and 99% conversion (by ³¹P NMR). The product was not isolated. ¹H NMR (D₂O, 500 MHz) δ 3.89 (m, 2H), 3.72 (d, J=11.6 Hz, 1 H), 3.65 (d, J=11.6 Hz, 1H), 2.93 (s, 1H). ¹³C NMR (D₂O, 126 MHz) δ 82.9 (s), 75.1 (s), 71.0 (d, J=6.9 Hz), 67.0 (d, J=4.5 Hz), 64.7 (s). 31P NMR (D₂O, 202 MHz) δ 3.39. HRMS: (ESI, m/z): calculated for [M−1]⁻C₅H₈O₆P: 195.0058; Found 195.0068 [M⁻H]⁻: 195.0058.

Method G2: Acetate Kinase: ATP-regeneration System Using Enzyme SEQ. ID No.: 20 and enzyme SEQ. ID No.: 21

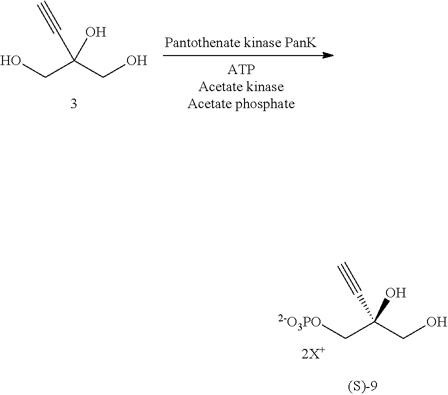

To a jacketed reactor aqueous solution 2-ethynyl-propane-1,2,3-triol (3) (11.47 kg, 8.7% wt, 8.61 mol) and water (7.5 kg) was charged, followed by 1M BIS-TRIS methane buffer pH 6.5 (1L) and magnesium chloride (41.4 g). ATP (48 g, 0.086 mol, 0.01 equivalent) and diammonium acetyl phosphate (2.021 kg, 89%, 10.33 mmol) were added, the solution was warmed up to 20° C. and the pH of the solution was re-adjusted to 6.8 using KOH (270.4 g). Evolved pantothenate kinase SEQ. ID No.: 20 (20.4 g) and evolved acetate kinase SEQ. ID No.: 21 (3 g) were then charged as solids. The reaction was stirred for at 20° C. for 16 h during which pH dropped to 5.5. Quantitative conversion of 2-ethynyl-propane-1,2,3-triol (3) was obtained as judged by ¹H and 31P NMR. Such prepared (S)-2-ethynyl-propane-1,2,3-triol 1-phosphate (9) solution (397 mM, 22.5 kg, 98% yield) was used in subsequent oxidation step without any further purification. ¹H NMR (D₂O, 500 MHZ) § 3.89 (m, 2H), 3.72 (d, J=11.6 Hz, 1 H), 3.65 (d, J=11.6 Hz, 1H), 2.93 (s, 1H).

Method G3: Acetate kinase: ATP-regeneration system using enzyme SEQ. ID No.: 20 and enzyme SEQ. ID No.: 21 with deuterated compound (3) to assign absolute stereochemistry and demonstrate desymmetrizing phosphorylation.

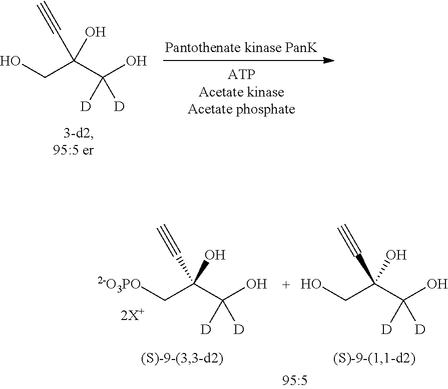

Evolved pantothenate kinase SEQ. ID No.: 20 (100 μL of 10 g/L solution in water) and evolved acetate kinase SEQ. ID No.: 21 (100 μL of 2 g/L solution in water) were added to a solution containing diammonium acetyl phosphate (41 mg), 2-ethynyl-propane-1,1-d2-1,2,3-triol ((R)-3-d2, 20 mg, 170 μmol), magnesium chloride (10 μL of 1 M solution in water), ADP (10 μL of 100 g/L solution in water), and sodium phosphate buffer (10 μL of 1 M solution in water) in water (800 μL) at pH 6.5. The reaction was incubated for 24 h at rt to give deuterated 2-ethynyl-propane-1,2,3-triol 1-phosphate analogs (S)-9-(3,3-d2) and (S)-9-(1,1-d2) in 95:5 ratio and 99% overall yield. The ratio of phosphorylated compounds was determined by ³¹P NMR to be ˜95:5, confirming stereoselective phosphorylation of the 2-ethynyl-propane-1,2,3-triol (3) at the pro-(S) hydroxyl group (i.e. a desymmetrizing phosphorylation). ¹H NMR (D₂O, 500 MHz) 8 3.89 (m, 2H), 3.72 (d, J=11.6 Hz, 1 H), 3.65 (d, J=11.6 Hz, 1H), 2.93 (s, 1H). ¹³C NMR (D₂O, 126 MHz) δ 82.9 (s), 75.1 (s), 71.0 (d, J=6.9 Hz), 67.0 (d, J=4.5 Hz), 64.7 (s).

Method G4: Acetate kinase: ATP-regeneration system using immobilized enzymes SEQ. ID No.: 20 and enzyme SEQ. ID No.: 21

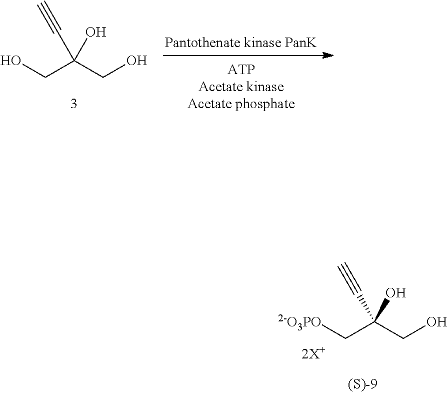

### Enzyme Immobilization Procedure:

Nuvia IMAC Ni-charged resin (75 mL based on settled volume) was added to a filter funnel and washed with water (9 column volumes, 3×225 mL) and binding buffer (1 column volume, 75 mL; 500 mM sodium chloride, 50 mM sodium phosphate, 15 mM imidazole, pH 8.0). In a vessel pantothenate kinase (SEQ ID NO.: 20, 6.0 g) lyophilized powder was resuspended in binding buffer (200 mL) and the washed resin was added. The solution was mixed using rotating mixer at 25° C. for 6 h. The resin was filtered and washed with binding buffer (6 column volumes, 6×225 mL) and BIS-TRIS buffer (8 column volumes, 600 mL; 50 mM, pH 6.2).

### Reaction Procedure:

An aqueous solution of 2-ethynyl-propane-1,2,3-triol (3) (574 g, 8.7% wt, 0.430 mol) and water (350 mL) was charged to a jacketed reactor, followed by IM BIS-TRIS methane buffer pH 6.5 (50 mL) and magnesium chloride (2.033 g, 0.01 mol). ATP (2.37 g, 0.0043 mol, 0.01 equivalent) and diammonium acetyl phosphate (101 g, 89%, 0.530 mmol, 1.2 eq) were added, the solution was warmed up to 20° C. and the pH of the solution was re-adjusted to 6.8 using 5 M KOH. Resin with immobilized pantothenate kinase SEQ. ID No.: 20 (25 mL) and evolved acetate kinase SEQ. ID No.: 21 (0.15 g) were then charged as solids. The reaction was stirred for at 20° C. for 16 h during which the pH dropped to 5.5. Quantitative conversion of 2-ethynyl-propane-1,2,3-triol (3) to (S)-2-ethynyl-propane-1,2,3-triol 1-phosphate (9) was obtained as judged by ¹H and ³¹P NMR. ¹H NMR (D₂O, 500 MHZ) δ 3.89 (m, 2H), 3.72 (d, J=11.6 Hz, 1 H), 3.65 (d, J=11.6 Hz, 1H), 2.93 (s, 1H).

Alternate Preparations of (R)-2-ethynyl-glyceraldehyde 3-phosphate (5): Method H1: Immobilized Galactose Oxidases SEQ ID No.: 16

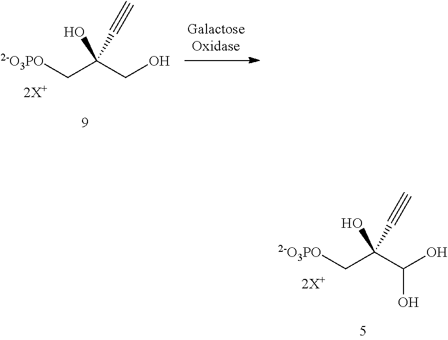

### Enzyme Immobilization Procedure:

Nuvia IMAC Ni-charged resin (10 mL based on settled volume) was added to a filter funnel and washed with binding buffer (10 column volumes, 100 mL; 500 mM sodium chloride, 50 mM sodium phosphate, 15 mM imidazole, pH 8.0) to remove the resin storage solution and give 16 g of washed resin. In a vessel evolved galactose oxidase (SEQ ID NO.: 16, 750 mg) lyophilized powders were resuspended in copper (II) sulphate solution (100 μM; 5.00 mL), followed by addition of binding buffer (20 mL) and the washed resin (3.0 g). The solution was mixed using rotating mixer at 20° C. for 5 h. The resin was filtered and washed with binding buffer (10 column volumes, 100 mL) and BIS-TRIS buffer (10 column volumes, 100 mL; 50 mM, pH 7.5) and it was used directly in the glycosylation reaction.

### Reaction Procedure:

The resin with immobilized galactose oxidase SEQ ID NO.: 16 (3.0 g) was added to a solution of (S)-2-ethynyl-propane-1,2,3-triol 1-phosphate (9, 5.4 mmol, 270 mM, 20 mL) in BIS-TRIS methane buffer (35 mM, pH adjusted to 7.2), followed by addition of copper (II) sulphate solution in water (30 μL, 100 mM) and horseradish peroxidase (PEO-301, 18 mg) and bovine catalase (C1345, 120 mg) resuspended in water (600 μL). The reaction was sealed with gas permeable membrane and shaken vigorously at 22° C. for 4 days to reach final conversion of 77% and give (R)-2-ethynyl-glyceraldehyde 3-phosphate (5) in 95% e.e. The enzyme resin was filtered off and the solution of the(R)-2-ethynyl-glyceraldehyde 3-phosphate (5) was used directly in the glycosylation reaction. ¹H NMR (D₂O, 400 MHZ): δ 5.02 (s, 1H), 4.00 (dq, 2 H), 2.88 (s, 1H). LC-MS: (ES, m/z): calculated for C₅H₇O₆P (M⁻H): 193.1; found 193.0.

### Method H2: Immobilized Galactose Oxidases SEQ ID No.: 17

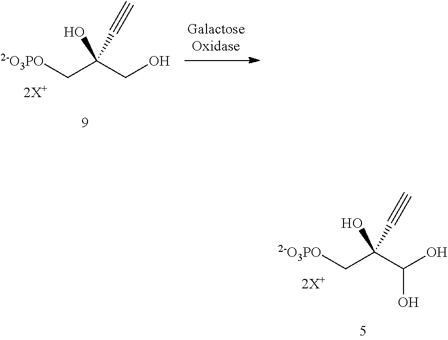

### Enzyme Immobilization Procedure:

Nuvia IMAC Ni-charged resin (10 mL based on settled volume) was added to a filter funnel and washed with binding buffer (10 column volumes, 100 mL; 500 mM sodium chloride, 50 mM sodium phosphate, 15 mM imidazole, pH 8.0) to remove the resin storage solution and give 16 g of washed resin. In a vessel, evolved galactose oxidase (SEQ ID NO.: 16, 750 mg) lyophilized powders were resuspended in copper (II) sulphate solution (100 μM; 5.00 mL), followed by addition of binding buffer (20 mL) and the washed resin (3.0 g). The solution was mixed using rotating mixer at 20° C. for 5 h. The resin was filtered and washed with binding buffer (10 column volumes, 100 mL) and BIS-TRIS methane buffer (10 column volumes, 100 mL; 50 mM, pH 7.5) and it was used directly in the reaction.

Reaction Procedure:

The resin with immobilized evolved galactose oxidase SEQ ID NO.: 17 (3.0 g) was added to a solution of (S)-2-ethynyl-propane-1,2,3-triol 1-phosphate (9, 5.4 mmol, 270 mM, 20 mL) in BIS-TRIS methane buffer (35 mM, pH adjusted to 7.2), followed by addition of copper (II) sulphate solution in water (30 μL, 100 mM) and horseradish peroxidase (PEO-301, 18 mg) and bovine catalase (C1345, 120 mg) resuspended in water (600 μL). The reaction was sealed with gas permeable membrane and shaken vigorously at 22° C. for 4 days to reach final conversion of 77% and give (R)-2-ethynyl-glyceraldehyde 3-phosphate (5) in 95% e.e. The enzyme resin was filtered off and the solution of the (R)-2-ethynyl-glyceraldehyde 3-phosphate (5) was used directly in the glycosylation reaction. ¹H NMR (D₂O, 400 MHZ): δ 5.02 (s, 1H), 4.00 (dq, 2 H), 2.88 (s, 1H). LC-MS: (ES, m/z): calculated for C₅H₇O₆P (M⁻H): 193.1; found 193.0.

### Method H3: Immobilized Galactose Oxidases SEQ ID No.: 18

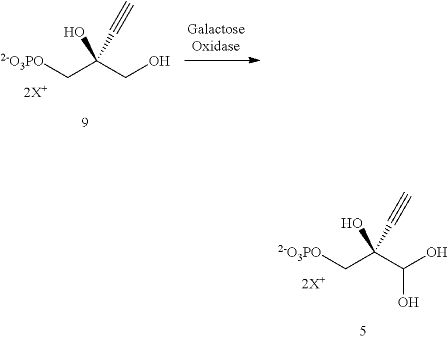

Enzyme Immobilization Procedure:

Nuvia IMAC Ni-charged resin (3 mL based on settled volume) was added to a filter funnel and washed with binding buffer (10 column volumes, 30 mL; 500 mM sodium chloride, 50 mM sodium phosphate, 15 mM imidazole, pH 8.0) to remove the resin storage solution and give 2.4 g of washed resin. In a vial evolved galactose oxidase (SEQ ID NO.: 18, 75 mg) lyophilized powders were resuspended in copper (II) sulphate solution (100 μM; 1.00 mL), followed by addition of binding buffer (5 mL) and the washed resin (400 mg). The solution was mixed using rotating mixer at 20° C. for 5 h. The resin was filtered and washed with binding buffer (10 column volumes, 4 mL) and BIS-TRIS methane buffer (10 column volumes, 4 mL; 50 mM, pH 7.5) and it was used directly in a reaction.

### Reaction Procedure:

Immobilized evolved GOase SEQ ID NO.: 18 was added (400 mg) to a solution of (S)-2-ethynyl-propane-1,2,3-triol 1-phosphate solution ((9), 5.4 mmol, 270 mM, 1 mL) in BIS-TRIS methane buffer (35 mM, pH adjusted to 7.2), , followed by addition of horseradish peroxidase (PEO-301, 1 mg) and catalase from Corynebacterium glutamicum (Roche, lyophilizate, #11650645103, 3 mg) resuspended in water (100 μL). The reaction was sealed with gas permeable membrane and shaken vigorously at 30° C. for 48 h. Final conversion after 2 days reached 90% conversion and the (R)-2-ethynyl-glyceraldehyde 3-phosphate (5) >99% e.e. The enzyme resin was filtered off and the solution of the (R)-2-ethynyl-glyceraldehyde 3-phosphate (5) was used directly without further purification. ¹H NMR (D₂O, 400 MHZ): δ 5.02 (s, 1H), 4.00 (dq, 2 H), 2.88 (s, 1H). LC-MS: (ES, m/z): calculated for C₅H₇O₆P (M⁻H): 193.1; found 193.0.

### Method H4: Immobilized Galactose Oxidases SEQ ID No.: 19

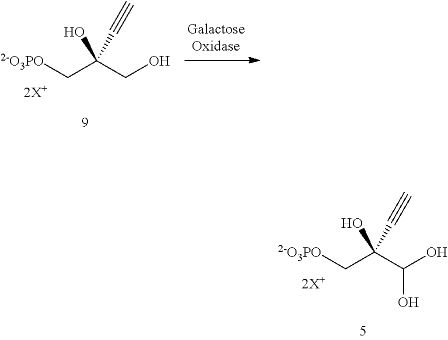

### Enzyme Immobilization Procedure:

Nuvia IMAC Ni-charged resin (3 mL based on settled volume) was added to a filter funnel and washed with binding buffer (10 column volumes, 30 mL; 500 mM sodium chloride, 50 mM sodium phosphate, 15 mM imidazole, pH 8.0) to remove the resin storage solution and give 2.4 g of washed resin. In a vial evolved galactose oxidase (SEQ ID NO.: 19, 75 mg) lyophilized powders were resuspended in copper (II) sulphate solution (100 μM; 1.00 mL), followed by addition of binding buffer (5 mL) and the washed resin (400 mg). The solution was mixed using rotating mixer at 20° C. for 5 h. The resin was filtered and washed with binding buffer (10 column volumes, 4 mL) and BIS-TRIS methane buffer (10 column volumes, 4 mL; 50 mM, pH 7.5) and it was used directly in a reaction.

### Reaction Procedure:

Immobilized evolved GOase SEQ ID NO.: 18 was added (400 mg) to a solution of (S)-2-ethynyl-propane-1,2,3-triol 1-phosphate solution (9, 5.4 mmol, 270 mM, 1 mL) in BIS-TRIS methane buffer (35 mM, pH adjusted to 7.2), , followed by addition of horseradish peroxidase (PEO-301, 1 mg) and catalase from Corynebacterium glutamicum (Roche, lyophilizate, #11650645103, 3 mg) resuspended in water (100 μL). The reaction was sealed with gas permeable membrane and shaken vigorously at 30° C. for 48 h. Final conversion after 2 days reached 100% conversion and (R)-2-ethynyl-glyceraldehyde 3-phosphate (5) was obtained in >99% e.e. The enzyme resin was filtered off and the solution of the (R)-2-ethynyl-glyceraldehyde 3-phosphate (5) was used directly without further purification. ¹H NMR (D₂O, 400 MHZ): δ 5.02 (s, 1H), 4.00 (dq, 2 H), 2.88 (s, 1H). LC-MS: (ES, m/z): calculated for C₅H₇O₆P (M⁻H): 193.1; found 193.0.

“Amino acids” are referred to herein by either their commonly known by the one-letter symbols recommended by IUPAC-IUB Biochemical Nomenclature Commission. For the purposes of the description herein, the codes used for the genetically encoded amino acids for the enzymes used in the methods herein are conventional in Table 2:

TABLE 2

| Amino acid | One letter code |
|---|---|
| alanine | A |
| arginine | R |
| asparagine | N |
| aspartic acid | D |
| asparagine or aspartic acid | B |
| cysteine | C |
| glutamic acid | E |
| glutamine | Q |
| glutamine or glutamic acid | Z |
| glycine | G |
| histidine | H |
| isoleucine | I |
| leucine | L |
| lysine | K |
| methionine | M |
| phenylalanine | F |
| proline | P |
| serine | S |
| threonine | T |
| tryptophan | W |
| tyrosine | Y |
| valine | V |

Sequence ID numbers for the enzymes employed, or that could be employed, in the process for synthesizing EFdA described herein and in the exemplified process steps in the Experimental Procedures described herein are provided, but not limited to, those in Table 3.

TABLE 3

| SEQ ID | |
|---|---|
| NO: | ENZYME AND AMINO ACID SEQUENCE |
| 1 | Galactose Oxidase (GOase) = Variant of Galactose Oxidase |
| | from Fusarium graminearum (formerly known as Dactylium |
| | dendroides) |
| | MASAPIGSAIPRNNWAVTCDSAQSGNECNKAIDGNKDTFWHTFYGANGDPKPP |
| | HTYTIDMKTTQNVNGLSVLPRQDGNQNGWIGRHEVYLSSDGTNWGSPVASGS |
| | WFADSTTKYSNFETRPARYVRLVAITEANGQPWTSIAEINVFQASSYTAPQPGL |
| | GRWGPTIDLPIVPAAAAIEPTSGRVLMWSSYRNDAFEGSPGGITLTSSWDPSTGI |
| | VSDRTSTVTKHDMFCPGISMDGNGQIVVDETATGGNDAKKTSLYDSSSDSWIP |
| | GPDMQVARGYQSSATMSDGRVFTIGGSFSGGRVEKNGEVYSPSSKTWTSLPNA |
| | KVNPMLTADKQGLYRSDNHAWLFGWKKGSVFQAGPSTAMNWYYTSGSGDV |
| | KSAGKRQSNRGVAPDAMCGNAVMYDAVKGKILTFGGSPDYEDSDATTNAHIIT |
| | LGEPGTSPNTVFASNGLYFARTFHTSVVLPDGSTFITGGQRRGIPTEDSTPVFTPE |
| | IYVPEQDTFYKQNPNSIVRAYHSISLLLPDGRVFNGGGGLCGDCTTNHFDAQIFT |
| | PNYLYDSNGNLATRPKITRTSTQSVKVGGRITISTDSSISKASLIRYGTATHTVNT |
| | DQRRIPLTLTNNGGNSYSFQVPSDSGVALPGYWMLFVMNSAGVPSVASTIRVT |
| | QGGGGSWSHPQFEK |
| 2 | Pantothenate Kinase (PanK) = Variant of Pantothenate |
| | Kinase from E. coli |
| | MSIKEQTLMTPYLQFDRNQWAALRDSVPMTLSEDEIARLKGINEDLSLEEVAEI |
| | YLPLSRLLNFYISSNLRRQAVLEQFLGTNGQRIPYIISIAGSVAVGKSTTARVLQA |
| | LLSRWPEHRRVELITTDGFLHPNQVLKERGLMKKKGFPESYDMHRLVKFVSDL |
| | KSGVPNVTAPVYSHLIYDVIPDGDKTVVQPDILILEGLNVLQSGMDYPHDPHHV |
| | FVSDFVDFSIYVDAPEDLLQTWYINRFLKFREGAFTDPDSYFHNYAKLTKEEAIK |
| | TAMTIWKEMNWLNLKQNILPTRERASLILTKSANHAVEEVRLRK |
| 3 | Acetate Kinase (AcK) = wild type Acetate Kinase from |
| | Thermotoga maritima |
| | MGSHHHHHHGSRVLVINSGSSSIKYQLIEMEGEKVLCKGIAERIGIEGSRLVHRV |
| | GDEKHVIERELPDHEEALKLILNTLVDEKLGVIKDLKEIDAVGHRVVHGGERFK |
| | ESVLVDEEVLKAIEEVSPLAPLHNPANLMGIKAAMKLLPGVPNVAVFDTAFHQ |
| | TIPQKAYLYAIPYEYYEKYKIRRYGFHGTSHRYVSKRAAEILGKKLEELKIITCHI |
| | GNGASVAAVKYGKCVDTSMGFTPLEGLVMGTRSGDLDPAIPFFIMEKEGISPQE |
| | MYDILNKKSGVYGLSKGFSSDMRDIEEAALKGDEWCKLVLEIYDYRIAKYIGA |
| | YAAAMNGVDAIVFTAGVGENSPITREDVCSYLEFLGVKLDKQKNEETIRGKEGI |
| | ISTPDSRVKVLVVPTNEELMIARDTKEIVEKIGR |
| 4 | Pyruvate Oxidase (PO) = wild type Pyruvate oxidase from |
| | Streptococcus thermophilus |
| | MGSSHHHHHHSSGLVPRGSHMTVGKTKVSTASLKVLAGWGIDTIYGIPSGTLA |
| | PLMEALGEQEETDIKFLQVKHEEVGAMAAVMQWKFGGKLGVCVGSGGPGAS |
| | HLINGLYDAAMDNTPVLAILGSPPQRELNMDAFQELNQNPMYDHIAVYNRRVA |
| | YAEQLPKLIDDAIRTAISKRGVAVLEVPGDFGYKEIANDAFYSSGHSYRDYVSS |
| | AINEVDIDAAVEVLNKSKRAVIYAGIGTMGHGPAVQELSRKIKAPIITTAKNFET |
| | FDYDFEGLTGSTYRVGWKPANEAVKEADTVLFVGSNFPFAEVEGTFSNVENFIQ |
| | IDNNPTMLGKRHNADVAILGDAGEAVQMLLEKVAPVEESAWWNANLKNIQN |
| | WRDYMTKLETKENGPLQLYQVYNAINKYADEDAIYSIDVGNTTQTSIRHLHMT |
| | PKNMWRTSPLFASMGIALPGGIGAKNVYPERQVFNLMGDGAFSMNYQDIVTN |
| | VRYNMPVINVVFTNTEYGFIKNKYEDTNTNTFGTEFTDVDYAMIGEAQGAVGF |
| | TVSRIEDMDQVMAAAVKANKEGKTVVIDAKITKDRPIPVETLKLDPALYSEEEI |
| | KAYKERYEAEELVPFSEFLKAEGLESKVAK |
| 5 | Deoxyribose-phosphate Aldolase (DERA) = wild type |
| | Deoxyribose-phosphate Aldolase from Shewanella halifaxensis |
| | MSDLKKAAQQAISLMDLTTLNDDDTDQKVIELCHKAKTPAGDTAAICIYPRFIPI |
| | ARKTLNEIGGDDIKIATVTNFPHGNDDIAIAVLETRAAVAYGADEVDVVFPYRA |
| | LMEGNETVGFELVKACKEACGEDTILKVIIESGVLADPALIRKASELSIDAGADFI |
| | KTSTGKVAVNATLEAAEIMMTVISEKNPKVGFKPAGGVKDAAAAAEFLGVAA |
| | RLLGDDWATPATFRFGASSLLTNLLHTLELADAPQGAQGY |
| 6 | Deoxyribose-phosphate Aldolase (DERA) = Variant of |
| | Deoxyribose-phosphate Aldolase (DERA) from Shewanella |
| | halifaxensis |
| | MCDLKKAAQRAISLMDLTTLNDDDTDQKVIELCHKAKTPAGDTAAIVIYPRFIPI |
| | ARKTLNEIGGLDIKIVTVTNFPHGNDDIAIAVLETRAAVAYGADEVDVVFPYRA |
| | LMEGNETVGFELVKACKEACGEDTILKVIIESGVLKDPALIRKASEISIDAGADFI |
| | KTSTGKVAVNATLEAAEIIMTVISEKNPKVGFKPAGGIKDAAAAAEFLGVAARL |
| | LGDDWATPATFRFGATDLLTNLLHTLELADAPQGAQGY |
| 7 | Sucrose phosphorylase (SP) = wild type Sucrose phosphorylase |
| | from Alloscardovia omnicolens |
| | MKNKVQLITYADRLGDGTLKSMTETLRKHFEGVYEGVHILPFFTPFDGADAGF |
| | DPVDHTKVDPRLGSWDDVAELSTTHDIMVDTIVNHMSWESEQFQDVMAKGED |
| | SEYYPMFLTMSSIFPDGVTEEDLTAIYRPRPGLPFTHYNWGGKTRLVWTTFTPQ |
| | QVDIDTDSEMGWNYLLSILDQLSQSHVSQIRLDAVGYGAKEKNSSCFMTPKTF |
| | KLIERIKAEGEKRGLETLIEVHSYYKKQVEIASKVDRVYDFAIPGLLLHALEFGK |
| | TDALAQWIDVRPNNAVNVLDTHDGIGVIDIGSDQMDRSLAGLVPDEEVDALVE |
| | SIHRNSKGESQEATGAAASNLDLYQVNCTYYAALGSDDQKYIAARAVQFFMPG |
| | VPQVYYVGALAGSNDMDLLKRTNVGRDINRHYYSAAEVASEVERPVVQALNA |
| | LGRFRNTLSAFDGEFSYSNADGVLTMTWADDATRATLTFAPKANSNGASVARL |
| | EWTDAAGEHATDDLIANPPVVA |
| 8 | Phosphopentomutase (PPM) = Variant of Phosphopentomutase |
| | from E. coli |
| | MKRAFIMVLDSFGIGATEDAERFGDVGADTLGHIAEACAKGEADNGRKGPLNL |
| | PNLTRLGLAKAHEGSTGFIPAGMDGNAEVIGAYAWAHEMSSGKDSVSGHWEI |
| | AGVPVLFEWGYFSDHENSFPQELLDKLVERANLPGYLGNCRSSGTVILDQLGEE |
| | HMKTGKPIFYTSAASVFQIACHEETFGLDKLYELCEIAREELTNGGYNIGRVIAR |
| | PFIGDKAGNFQRTGNRRDLAVEPPAPTVLQKLVDEKHGQVVSVGKIADIYANC |
| | GITKKVKATGLDALFDATIKEMKEAGDNTIVFTNFVDFDSSWGHRRDVAGYAA |
| | GLELFDRRLPELMSLLRDDDILILTADHGCDPTWTGTDHTREHIPVLVYGPKVK |
| | PGSLGHRETFADIGQTLAKYFGTSDMEYGKAMF |
| 9 | Purine Nucleoside Phosphorylase (PNP) = Variant of Purine |
| | Nucleoside Phosphorylase from E. coli |
| | MATPHINAEMGDFADVVLMPGDPLRAKYIAETFLEDAREVNNVRGMLGFTGT |
| | YKGRKISVMGHGAGIPSCSIYTKELITDFGVKKIIRVGSCGAVLPHVKLRDVVIG |
| | MGACTDSKVNRIRFKDHDFAAIADFDMVRNAVDAAKALGIDARVGNLFSADL |
| | FYSPDGEMFDVMEKYGILGVEMEAAGIYGVAAEFGAKALTICTVSDHIRTHEQ |
| | TTAAERQTTFNDMIKIALESVLLGDKE |
| 10 | Galactose Oxidase (GOase) = Variant of Galactose Oxidase |
| | from Fusarium graminearum (formerly known as Dactylium |
| | dendroides) |
| | MASAPIGVAIPRNNWAVTCDSAQSGNECNKAIDGNKDTFWHTQYGVNGDPKP |
| | PHTITIDMKTVQNVNGLSVLPRQDGNQNGWIGRHEVYLSSDGVNWGSPVASGS |
| | WFADSTTKYSNFETRPARYVRLVAITEANGQPWTSIAEINVFQASSYTAPQPGL |
| | GRWGPTIDLPIVPSAAAIEPTSGRVLMWSSYRQDAFEGSPGGITLTSSWDPSTGI |
| | VSDRTSTVTKHDMFCPGISMDGNGQIVVSGGNDAKKTSLYDSSSDSWIPGPDM |
| | QVARGYQSSATMSDGRVFTIGGSFSGGQVEKNGEVYSPSSKTWTSLPNAKVNP |
| | MLTADKQGLYRSDNHAWLFGWKKGSVFQAGPSTAMNWYYTSGSGDVKSAG |
| | KRQSNRGVAPDAMCGNAVMYDAVKGKILTFGGSPDYEDSDATTNAHIITLGEP |
| | GTSPNTVFASNGLYFARTFHTSVVLPDGSTFITGGQQRGIPTEDSTPVFTPEIYVP |
| | EQDTFYKQNPNSIVRAYHSISLLLPDGRVFNGGGGLCGDCTTNHFDAQIFTPNY |
| | LYDSNGNLATRPKITRTSTQSVVVGGWITIWTDMSISAASLIRYGTATHTVNTD |
| | QRRIPLTLTNNGGNSYSFQVPSDSGVALPGYWMLFVMNSAGVPSVASTIRVTQ |
| | GQTGHHHHHH |
| 11 | Galactose Oxidase (GOase) = Variant of Galactose Oxidase |
| | from Fusarium graminearum (formerly known as Dactylium |
| | dendroides) |
| | MASAPIGVAIPRNNWAVTCDSAQSGNECNKAIDGNKDTFWHTQYGVNGDPKP |
| | PHTITIDMKTVQNVNGLSVLPRQDGNQNGWIGRHEVYLSSDGVNWGSPVASGS |
| | WFADSTTKYSNFETRPARYVRLVAITEANGQPWTSIAEINVFQASSYTAPQPGL |
| | GRWGPTIDLPIVPSAAAIEPTSGRVLMWSSYRQDAFEGSPGGITLTSSWDPSTGI |
| | VSDRTSTVTGHDMFCPGISMDGNGQIVVSGGNDAKKTSLYDSSSDSWIPGPDM |
| | QVARGYNSSATMSDGRVFTIGGSFSGGQVEKNGEVYSPSSKTWTSLPNAKVNP |
| | MLTADKQGLYRSDNHAWLFGWKKGSVFQAGPSTAMNWYYTSGSGDVKSAG |
| | KRQSNRGVAPDAMCGNAVMYDAVKGKILTFGGSPDYQDSDATTNAHIITLGEP |
| | GTSPNTVFASNGLLFARTFHTSVVLPDGSTFITGGQQRGIPTEDSTPVFTPEIYVP |
| | EQDTFYKQNPNSIVRAYHSISLLLPDGRVFNGGGGLCGDCETNHFDAQIFTPNY |
| | LYDSNGNLATRPKITRTSTQSVVVGGWITIWTDMSISAASLIRYGTATHTVNTD |
| | QRRIPLTLTNNGGNSYSFQVPSDSGVALPGYWMLFVMNSAGVPSVASTINVTQ |
| | GQTGHHHHHH |
| 12 | Pantothenate Kinase (PanK) = Variant of Pantothenate |
| | Kinase from E. coli |
| | MSIKEQTLMTPYLQLDRNQWAALRDSNPMTLSEDEIARLKGINEDLSLEEVAEV |
| | YLPLSRLLNFYISSNLRRQAVLEQFLGTNGQRIPYIISIAGSVAVGKSTTARVLQA |
| | LLSRWPEHRRVELITTDGFLHPNQVLKERGLMKKKGFPESYDMHRLMKFVKDL |
| | KSGVPNVTAPVYSHLIYDVIPDGDKTVVQPDILILEGLNVLQSGMDYPHDPHHV |
| | FVSDFVDFSIYVDAPEDLLQTWYINRFLKFREGAFTDPDSYFHGYAKLTKEEAIK |
| | TAMTIWKEMNHLNLKQNILPTRERASLILTKSANHIVEEVRLRK |
| 13 | Pantothenate Kinase (PanK) = Variant of Pantothenate |
| | Kinase from E. coli |
| | MHHHHHHGGMSIKEQTLMTPYLQLDRNQWAALRDSNPMTLSEDEIARLKGIN |
| | EDLSLEEVAEVYLPLSRLLNFYISSNLRRQAVLEQFLGTNGQRIPYIISIAGSVAV |
| | GKSTTARVLQALLSRWPEHRRVEHITTDGFLHPNQVLKERGLMGKKGFPESYD |
| | MHRLMKFVKDLKSGVPNVTAPVYSHLIYDVIPDGDKTVVQPDILILEGLNVLQS |
| | GMDYPHDPHHVFVSDFVDFSIYVDAPEDLLQTWYINRFLKFREGAFTDPDSYFH |
| | GYAKLTKEEAIKTAMTIWKEMNHLNLKQNILPTRERASLILTKSANHIVEEVRL |
| | RK |
| 14 | Deoxyribose-phosphate Aldolase (DERA) = Variant of |
| | Deoxyribose-phosphate Aldolase (DERA) from Shewanella |
| | halifaxensis |
| | MHHHHHHCDLKKAAQRAISLMDLTTLNDDDTDQKVIELCHKAKTPAGDTAAI |
| | VIYPRFIPIARKTLNEIGGLDIKIVTVTNFPHGNDDIAIAVLETRAAVAYGADEVD |
| | VVFPYRALMEGNETVGFELVKACKEACGEDTILKVIIESGVLKDPALIRKASEISI |
| | DAGADFIKTSTGKVAVNATLEAAEHMTVISEKNPKVGFKPAGGIKDAAAAAEF |
| | LGVAARLLGDDWATPATFRFGATDLLTNLLHTLELADAPQGAQGY |
| 15 | Purine Nucleoside Phosphorylase (PNP) = Variant of |
| | Purine Nucleoside Phosphorylase from E. coli |
| | MATPHINAEMGDFADVVLMPGDPLRAKYIAETFLEDAREVNNVRGMLGFTGT |
| | YKGRKISVMGHGMGIPSCSIYTKELITDFGVKKIIRVGSCGAVLPHVKLRDVVIG |
| | MGACTDSKVNRIRFKDHDFAAIADFDMVRNAVDAAKALGIDARVGNLFSADL |
| | FYSPDGEMFDVMEKYGILGVEMEAAGIYGVAAEFGAKALTICTVSDHIRTHEQ |
| | TTAAERQTTFNDMIKIALESVLLGDKE |
| 16 | Galactose Oxidase (GOase) = Variant of Galactose Oxidase |
| | from Fusarium graminearum (formerly known as Dactyhum |
| | dendroides) |
| | MASAPIGVAIPRNNWAVTCDSAQSGNECNKAIDGNKDTFWHTQYGVNGDPKP |
| | PHTITIDMKTVQNVNGLSVLPRQDGNQNGWIGRHEVYLSSDGVNWGSPVASGS |
| | WFADSTTKYSNFETRPARYVRLVAITEANGQPWTSIAEINVFQASSYTAPQPGL |
| | GRWGPTIDLPIVPSAAAIEPTSGRVLMWSSYRQDAFEPSPGGITLTSSWDPSTGIV |
| | SDRTSTVTGHDMFCPGISMDGNGQIVVSGGNDAKKTSLYDSSSDSWIPGPDMQ |
| | VARGYNSSATMSDGRVFTIGGSYSGGQVEKNGEVYSPSSKTWTSLPNAKVNPM |
| | LTADKQGLYRSDNHAWLFGWKKGSVFQAGPSTAMNWYYTSGSGDVKSAGKR |
| | QSDRGVAPDAMCGNAVMYDAVKGKILTFGGSPDYQDSDATTNAHIITLGEPGT |
| | SPNTVFASNGLLFARTFHTSVVLPDGSVFITGGQQRGVPLEDSTPVFTPEIYVPEQ |
| | DTFYKQNPNSIVRAYHSISLLLPDGRVFNGGGGLCGDCETNHFDAQIFTPNYLY |
| | DSNGNLATRPKITRTSTQSVVVGGWITIWTDMSISAASLIRYGTATHTVNTDQR |
| | RIGLTLTNNGGNSYSFQVPSDSGVALPGYWMLFVMNSAGVPSVASTINVTQGQ |
| | TGHHHHHH |
| 17 | Galactose Oxidase (GOase) = Variant of Galactose Oxidase |
| | from Fusarium graminearum (formerly known as Dactylium |
| | dendroides) |
| | MASAPIGVAIPRNNWAVTCDSAQSGNECIKAIDGNKDTFWHTQYGVNGDPKPP |
| | HTITIDMKTVQNVNGLSVLPRQDGNQNGWIGRHEVYLSSDGVNWGSPVASGS |
| | WFADSTTKYSNFETRPARYVRLVAITEANGQPWTSIAEINVFQASSYTAPQPGL |
| | GRWGPTIDLPIVPSAAAIEPTSGRVLMWSSYRQDAFEDSPGGITLTSSWDPSTGI |
| | VSDRTSTVTGHDMFCPGISMDGNGQIVVSGGNDAKKTSLYDSSSDSWIPGPDM |
| | QVARGYNSSATMSDGRVFTIGGSYSGGQVEKNGEVYSPSSKTWTSLPNAKVNP |
| | MLTADKQGLYRSDNHAWLFGWKKGSVFQAGPSTAMNWYYTSGSGDVKSAG |
| | KRQSDRGVAPDAMCGNAVMYDAVKGKILTFGGSPDYQDSDATTNAHIITLGEP |
| | GTSPNTVFASNGLLFARTFHTSVVLPDGSVFITGGQQRGVPLEDSTPVFTPEIYVP |
| | EQDTFYKQNPNSIVRAYHSISLLLPDGRVFNGGGGLCGDCETNHFDAQIFTPNY |
| | LYDSNGNLATRPKITRTSTQSVVVGGWITIWTDMSISAASLIRYGTATHTVNTD |
| | QRRIGLTLTNNGGNSYSFQVPSDSGVALPGYWMLFVMNSAGVPSVASTINVTQ |
| | GQTGHHHHHH |
| 18 | Galactose Oxidase (GOase) = Variant of Galactose Oxidase |
| | from Fusarium graminearum (formerly known as Dactylium |
| | dendroides) |
| | MASAPIGVAIPRNNWAVTCDSAQSGNECIKAIDGNKDTFWHTQYGVNGDPKPP |
| | HTITIDMKTVQNVNGLSVLPRQDGNQNGWIGRHEVYLSSDGVNWGSPVASGS |
| | WFADSTTKYSNFETRPARYVRLVAITEANGQPWTSIAEINVFQASSYTAPQPGL |
| | GRWGPTIDLPIVPSAAAIEPTSGRVLMWSSYRQDAFEDSPGGITLTSSWDPSTGI |
| | VSDRTSTVTGHDMFCPGISMDGNGQIVVSGGNDAKKTSLYDSSSDSWIPGPDM |
| | QVARGYNSSATMSDGRVFTIGGSYSGGQVEKNGEVYSPSSKTWTSLPNAKVNP |
| | MLTADKRGLYRSDNHAWLFGWKKGSVFQAGPSTAMNWYYTSGSGDVKSAG |
| | KRQSDRGVAPDAMCGNAVMYDAVKGKILTFGGSPDYQDSDATTNAHIITLGEP |
| | GTSPNTVFASNGLLFARTFHTSVVLPDGSVFITGGQQRGVPLEDSTPVFTPEIYVP |
| | EQDTFYKQNPNSIVRAYHSISLLLPDGRVFNGGGGLCGDCETNHFDAQIFTPNY |
| | LYDSNGNLATRPKITRTSTQSVVVGGWITIWTDMSISAASLIRYGTATHTVNTD |
| | QRRIGLTLTNNGGNSYSFQVPSDSGVALPGYWMLFVMNSAGVPSVASTINVTQ |
| | GQTGHHHHHH |
| 19 | Galactose Oxidase (GOase) = Variant of Galactose Oxidase |
| | from Fusarium graminearum (formerly known as Dactylium |
| | dendroides) |
| | MASAPIGVAIPRNNWAVTCDSAQSGNECIKAIDGNKDTFWHTQYGVNGDPKPP |
| | HTITIDMKTVQNVNGLSVLPRQDGNQNGWIGRHEVYLSSDGVNWGSPVASGS |
| | WFADSTTKYSNFETRPARYVRLVAITEANGQPWTSIAEINVFQASSYTAPQPGL |
| | GRWGPTIDLPIVPSAAAIEPTSGRVLMWSSYRQDAFRDSPGGITLTSSWDPSTGI |
| | VSDRTSTVTGHDMFCPGISMDGNGQIVVSGGNDAKKTSLYDSSSDSWIPGPDM |
| | QVARGYNSSATMSDGRVFTIGGSYSGGQVEKNGEVYSPSSKTWTSLPNAKVNP |
| | MLTADKQGLYRSDNHAWLFGWKKGSVFQAGPSTAMNWYYTSGSGDVKSAG |
| | KRQSDRGVAPDAMCGNAVMYDAVKGKILTFGGSPDYQDSDATTNAHIITLGEP |
| | GTSPNTVFASNGLLFARTFHTSVVLPDGSVFITGGQQRGVPLEDSTPVFTPEIYVP |
| | EQDTFYKQNPNSIVRAYHSISLLLPDGRVFNGGGGLCGDCETNHFDAQIFTPNY |
| | LYDSNGNLATRPKITRTSTQSVVVGGWITIWTDMSISAASLIRYGTATHTVNTD |
| | QRRIGLTLTNNGGNSYSFQVPSDSGVALPGYWMLFVMNSAGVPSVASTINVTQ |
| | GQTGHHHHHH |
| 20 | Pantothenate Kinase (PanK) = Variant of Pantothenate Kinase |
| | from E. coli |
| | MHHHHHHGGSGSIKEQTLMTPYLQLDRNQWAALRDSNPMTLSEDEIARLKGIN |
| | EDLSLEEVAEVYLPLSRLLNFYISSNLRRQAQLEQFLGTNGQRIPYIISIAGSVAV |
| | GKSTFARVLQALLSRWPEHRRVEHITTDGFLHPNQVLKERGLMGKKGFPESYD |
| | MHRLMKFVKDLKSGVPNVTAPVYSHLIYDVIPDGDKTVVQPDILILEGLNVLQS |
| | GMDYPHDPHHVFVSDFVDFSIYVDAPEDLLQTWYINRFLKFREGAFTDPDSYFH |
| | GYAKLTKEEAIKTAMTIWKEMNHVNLKQNILPTRERASLILTKSANHIVEEVRL |
| | RK |
| 21 | Acetate Kinase (AcK) = Variant of Acetate Kinase from |
| | Thermotoga maritima |
| | MGSHHHHHHGSRVLNINSGSSSIKYQLIEMEGEKVLCKGIAERIGIEGSRLVHRV |
| | GDEKHVIERELPDHEEALKLILNTLVDEKLGVIKDLKEIDAVGHRVVHGGERFK |
| | ESVLVDEEVLKAIEEVSPLAPLHNPANLMGIKAAMKLLPGVPNVQVFDTAFHQ |
| | TIPQKAYLYAIPYEYYEKYKIRRYGFHGISHRYVSKRAAEILGKKLEELKIITCHI |
| | GNGASVAAVKYGKCVDTSMGFTPLEGLVMGTRSGDLDPAIPFFIMEKEGISPQE |
| | MYDILNKKSGVYGLSKGFSSDMRDNLEAALKGDEWCKLVLEIYDYRIAKYIGA |
| | YAAAMNGVDAIVFTAGVGENSPITREDVCKYLEFLGVKLDKQKNEETIRGKEGI |
| | ISTPDSRVKVLVVPTNEELMIARDTKEIVEKIGR |

Horseradish Peroxidase: wild type peroxidase from horseradish Type I, commercially available from SIGMA (P8125), isolated from horseradish roots (Amoracia rusticana).

Catalase: (1) wild type Catalase from bovine liver, commercially available from SIGMA (C1345); or (2) CAT-101, Biocatalytics; or (3) from Corynebacterium glutamicum(Roche, #11650645103).

Additional embodiments of this invention include, but are not limited to, the use of the following enzymes in the synthetic process steps described herein for producing a 4′-ethynyl 2′-deoxy nucleoside or an analog thereof, for example, EFdA.
A. A purine nucleoside phosphorylase.

1A. An engineered purine nucleoside phosphorylase comprising a polypeptide sequence having at least 85%, 86%, 87%, 88%, 89%, 90%, 91%, 92%, 93%, 94%, 95%, 96%, 97%, 98%, 99% or more sequence identity to SEQ ID NO.: 9 or SEQ ID NO.: 15, or a functional fragment thereof, wherein the polypeptide sequence of said engineered purine nucleoside phosphorylase comprises at least one amino acid substitution or amino acid substitution set as compared to SEQ ID NO: 9 or SEQ ID NO.: 15.

2A. The engineered purine nucleoside phosphorylase of 1A, wherein said engineered purine nucleoside phosphorylase comprises a polypeptide sequence that is at least 85%, 86%, 87%, 88%, 89%, 90%, 91%, 92%, 93%, 94%, 95%, 96%, 97%, 98%, 99% or more identical to SEQ ID NO: 9 or SEQ ID NO.: 15.

3A. An engineered purine nucleoside phosphorylase which is comprised of the polypeptide sequence as set forth in SEQ ID NO: 9 or SEQ ID NO.: 15.

4A. The engineered purine nucleoside phosphorylase of any one of 1A to 3A, which comprises at least one improved property compared to wild-type E. coli purine nucleoside phosphorylase.

5A. The engineered purine nucleoside phosphorylase of 4A, wherein said improved property comprises improved activity on substrate compound 6.5 (in its ring form or as an open chain aldehyde or hydrate, or a salt of any of the foregoing) as compared to wild type E. colipurine nucleoside phosphorylase.

6A. The engineered purine nucleoside phosphorylase of 4A, wherein said improved property comprises improved production of EFdA (compound 7) as compared to wild type E. coli purine nucleoside phosphorylase.

7A. The engineered purine nucleoside phosphorylase of any of one of 1A to 6A, wherein said engineered purine nucleoside phosphorylase is purified.

8A. The engineered purine nucleoside phosphorylase of any of one of 1A to 7A, wherein the at least one amino acid substitution (i.e., one or more amino acid substitution(s)) are conservative amino acid substitution(s).
B. A phosphopentomutase.

1B. An engineered phosphopentomutase comprising a polypeptide sequence having at least 85%, 86%, 87%, 88%, 89%, 90%, 91%, 92%, 93%, 94%, 95%, 96%, 97%, 98%, 99% or more sequence identity to SEQ ID NO.: 8, or a functional fragment thereof, wherein the polypeptide sequence of said engineered phosphopentomutase comprises at least one amino acid substitution or amino acid substitution set as compared to SEQ ID NO: 8.

2B. The engineered phosphopentomutase of 1B, wherein said engineered phosphopentomutase comprises a polypeptide sequence that is at least 85%, 86%, 87%, 88%, 89%, 90%, 91%, 92%, 93%, 94%, 95%, 96%, 97%, 98%, 99% or more identical to SEQ ID NO.: 8.

3B. An engineered phosphopentomutase which is comprised of the polypeptide sequence as set forth in SEQ ID NO.: 8.

4B. The engineered phosphopentomutase of any one of 1B to 3B, which comprises at least one improved property compared to wild-type E. coli phosphopentomutase.

5B. The engineered phosphopentomutase of 4B, wherein said improved property comprises improved activity on substrate compound 6 (in its ring form or as an open chain aldehyde or hydrate, or a salt of any of the foregoing) as compared to wild type E. coliphosphopentomutase.

6B. The engineered phosphopentomutase of 4B, wherein said improved property comprises improved production of compound 6.5 or compound 7 (EFdA) as compared to wild type E. coli phosphopentomutase.

7B. The engineered phosphopentomutase of any of one of 1B to 6B, wherein said engineered phosphopentomutase is purified.

8B. The engineered phosphopentomutase of any of one of 1B to 7B, wherein the at least one amino acid substitution (i.e., one or more amino acid substitution(s)) are conservative amino acid substitution(s).
C. A deoxyribose-phosphate aldolase.

1C. A deoxyribose-phosphate aldolase which is comprised of the wild type from Shewanella halifaxensis polypeptide sequence as set forth in SEQ ID NO.: 5.

2C. An engineered deoxyribose-phosphate aldolase which is comprised of the polypeptide sequence as set forth in SEQ ID NO.: 6 or SEQ ID NO.: 14.

3C. An engineered deoxyribose-phosphate aldolase, wherein said engineered deoxyribose-phosphate aldolase comprises a polypeptide sequence that is at least 85%, 86%, 87%, 88%, 89%, 90%, 91%, 92%, 93%, 94%, 95%, 96%, 97%, 98%, 99% or more identical to SEQ ID NO.: 5, SEQ ID NO.: 6 or SEQ ID NO.: 14.

4C. An engineered deoxyribose-phosphate aldolase comprising a polypeptide sequence having at least 85%, 86%, 87%, 88%, 89%, 90%, 91%, 92%, 93%, 94%, 95%, 96%, 97%, 98%, 99% or more sequence identity to SEQ ID NO.: 5, SEQ ID NO.: 6 or SEQ ID NO.: 14, or a functional fragment thereof, wherein the polypeptide sequence of said engineered deoxyribose-phosphate aldolase comprises at least one amino acid substitution or amino acid substitution set as compared to SEQ ID NO.: 5, SEQ ID NO.: 6 or SEQ ID NO.: 14.

5C. The deoxyribose-phosphate aldolase of any one of 1C to 4C, which has activity on substrate compound 5 ((R)-2-ethynyl-glyceraldehyde 3-phosphate, the hydrate thereof, or a salt of either of the foregoing).

6C. The deoxyribose-phosphate aldolase of any one of 1C to 5C, which comprises the ability to produce compound 6 (4-ethynyl-D-2-deoxyribose 5-phosphate, or the open chain aldehyde or hydrate form thereof, or a salt of any of the foregoing) without need for protecting groups on substrate compound 5 ((R)-2-ethynyl-glyceraldehyde 3-phosphate, the hydrate thereof, or a salt of either of the foregoing) during the reaction.

7C. The engineered deoxyribose-phosphate aldolase of any one of 2C to 6C, wherein the deoxyribose-phosphate aldolase has an improved property which comprises improved production of compound 6 (4-ethynyl-D-2-deoxyribose 5-phosphate, or the open chain aldehyde or hydrate form thereof, or a salt of any of the foregoing) as compared to wild-type Shewanella halifaxensis deoxyribose-phosphate aldolase.

8C. The deoxyribose-phosphate aldolase of any of one of 1C to 7C, wherein said deoxyribose-phosphate aldolase is purified.

9C. The engineered deoxyribose-phosphate aldolase of any of one of 2C to 7C, wherein the at least one amino acid substitution (i.e., one or more amino acid substitution(s)) are conservative amino acid substitution(s).
D. A pantothenate kinase.

1D. An engineered pantothenate kinase comprising a polypeptide sequence having at least 85%, 86%, 87%, 88%, 89%, 90%, 91%, 92%, 93%, 94%, 95%, 96%, 97%, 98%, 99% or more sequence identity to SEQ ID NO: 2, SEQ ID NO.: 12, SEQ ID NO.: 13 or SEQ ID NO.: 20, or a functional fragment thereof, wherein the polypeptide sequence of said engineered pantothenate kinase comprises at least one amino acid substitution or amino acid substitution set as compared to SEQ ID NO: 2, SEQ ID NO.: 12, SEQ ID NO.: 13 or SEQ ID NO.: 20.

2D. The engineered pantothenate kinase of 1D, wherein said engineered pantothenate kinase comprises a polypeptide sequence that is at least 85%, 86%, 87%, 88%, 89%, 90%, 91%, 92%, 93%, 94%, 95%, 96%, 97%, 98%, 99% or more identical to SEQ ID NO.: 2, SEQ ID NO.: 12, SEQ ID NO.: 13 or SEQ ID NO.: 20.

3D. An engineered pantothenate kinase, which is comprised of the polypeptide sequence as set forth in SEQ ID NO.: 2, SEQ ID NO.: 12, SEQ ID NO.: 13 or SEQ ID NO.: 20.

4D. The engineered pantothenate kinase of any one of 1D to 3D, which comprises at least one improved property compared to wild-type E. coli pantothenate kinase.

5D. The engineered pantothenate kinase of 4D, wherein said improved property comprises improved activity on substrate compound 4 ((R)-2-ethynyl-glyceraldehyde or hydrate form thereof) as compared to wild-type E. coli pantothenate kinase.

6D. The engineered pantothenate kinase of 5D, wherein said improved property comprises improved production of compound 5 ((R)-2-ethynyl-glyceraldehyde 3-phosphate), as compared to wild-type pantothenate kinase.

7D. The engineered pantothenate kinase of 4D, wherein said improved property comprises improved activity on substrate compound 3 (2-ethynyl-propane-1,2,3-triol) as compared to wild-type E. coli pantothenate kinase.

8D. The engineered pantothenate kinase of 7D, wherein said improved property comprises improved production of compound 9 ((S)-2-ethynyl-propane-1,2,3-triol 1-phosphate), as compared to wild-type pantothenate kinase.

9D. The engineered pantothenate kinase of any of one of 1D to 8D, wherein said pantothenate kinase is purified.

10D. The engineered pantothenate kinase of any of one of 1D to 9D, wherein the at least one amino acid substitution (i.e., one or more amino acid substitution(s)) are conservative amino acid substitution(s).
E. A galactose oxidase.

1E. An engineered galactose oxidase comprising a polypeptide sequence having at least 85%, 86%, 87%, 88%, 89%, 90%, 91%, 92%, 93%, 94%, 95%, 96%, 97%, 98%, 99% or more sequence identity to SEQ ID NOs.: 1, 10, 11, 16, 17, 18 or 19, or a functional fragment thereof, wherein the polypeptide sequence of said engineered galactose oxidase comprises at least one amino acid substitution or amino acid substitution set as compared to SEQ ID NOs.: 1, 10, 11, 16, 17, 18 or 19.

2E. The engineered galactose oxidase of 1E, wherein said engineered galactose oxidase comprises a polypeptide sequence that is at least 85%, 86%, 87%, 88%, 89%, 90%, 91%, 92%, 93%, 94%, 95%, 96%, 97%, 98%, 99% or more identical to SEQ ID NOs.: 1, 10, 11, 16, 17, 18 or 19.

3E. An engineered galactose oxidase which is comprised of the polypeptide sequence as set forth in SEQ ID NOs.: 1, 10, 11, 16, 17, 18 or 19.

4E. The engineered galactose oxidase of any one of 1E to 3E, which comprises at least one improved property compared to wild-type F. graminearum galactose oxidase. 5E. The engineered galactose oxidase of 4E, wherein said improved property comprises improved activity on a substrate which is a primary alcohol as compared to wild type F. graminearum galactose oxidase.

6E. The engineered galactose oxidase of 4E, wherein said improved property comprises improved activity on substrate compound 3 (2-ethynyl-propane-1,2,3-triol) as compared to wild type F. graminearum galactose oxidase.

7E. The engineered galactose oxidase of 6E, wherein said improved property comprises improved production of compound 4 ((R)-2-ethynyl-glyceraldehyde or hydrate form thereof) as compared to wild type F. graminearum galactose oxidase.

8E. The engineered galactose oxidase of 4E, wherein said improved property comprises improved activity on substrate compound 9 ((S)-2-ethynyl-propane-1,2,3-triol 1-phosphate), as compared to wild type F. graminearum galactose oxidase.

9E. The engineered galactose oxidase of 8E, wherein said improved property comprises improved production of compound 5 ((R)-2-ethynyl-glyceraldehyde 3-phosphate or hydrate form thereof), as compared to wild type F. graminearum galactose oxidase.

10E. The engineered galactose oxidase of any of one of 1E to 9E, wherein said galactose oxidase is purified.

11E. The engineered galactose oxidase of any of one of 1E to 10E, wherein the at least one amino acid substitution (i.e., one or more amino acid substitution(s)) are conservative amino acid substitution(s).
F. An acetate kinase.

1F. An acetate kinase, which is comprised of the wild type from Thermotoga maritima polypeptide sequence as set forth in SEQ ID NO.: 3 or SEQ ID NO.: 21. 2F. An engineered acetate kinase,, wherein said engineered acetate kinase comprises a polypeptide sequence that is at least 85%, 86%, 87%, 88%, 89%, 90%, 91%, 92%, 93%, 94%, 95%, 96%, 97%, 98%, 99% or more identical to SEQ ID NO.: 3 or SEQ ID NO.: 21. 3F An engineered acetate kinase comprising a polypeptide sequence having at least 85%, 86%, 87%, 88%, 89%, 90%, 91%, 92%, 93%, 94%, 95%, 96%, 97%, 98%, 99% or more sequence identity to SEQ ID NO.: 3 or SEQ ID NO.: 21, or a functional fragment thereof, wherein the polypeptide sequence of said engineered acetate kinase comprises at least one amino acid substitution or amino acid substitution set as compared to SEQ ID NO.: 3 or SEQ ID NO: 21.

4F. The acetate kinase of 2F or 3F, which comprises at least one improved property compared to wild-type T. maritima acetate kinase.

5F. The acetate kinase of 4F, wherein said improved property comprises improved activity for ATP-cofactor recycling in the phosphorylation reaction on substrate compound 4 ((R)-2-ethynyl-glyceraldehyde or hydrate form thereof) as compared to wild-type Thermotoga maritima acetate kinase.

6F. The acetate kinase of 5F, wherein said improved property comprises improved production of compound 5 ((R)-2-ethynyl-glyceraldehyde 3-phosphate or a hydrate form thereof or a salt of either of the foregoing) as compared to wild-type Thermotoga maritima acetate kinase.

7F. The acetate kinase of 4F, wherein said improved property comprises improved activity for ATP-cofactor recycling in the phosphorylation reaction on substrate compound 3 (2-ethynyl-propane-1,2,3-triol) as compared to wild-type Thermotoga maritima acetate kinase. 8F. The acetate kinase of 7F, wherein said improved property comprises improved production of compound 9 ((S)-2-ethynyl-propane-1,2,3-triol 1-phosphate or a salt of either of the foregoing) as compared to wild-type Thermotoga maritima acetate kinase.

9F. The acetate kinase of any of one of 1F to 8F, wherein said acetate kinase is purified.

10F. The engineered acetate kinase of any of one of 2F to 7F, wherein at least one amino acid substitution (i.e., one or more amino acid substitution(s)) are conservative amino acid substitution(s).

What is claimed is:

1. The compound

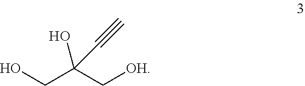

2. The compound

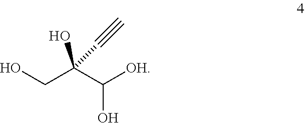

3. The compound 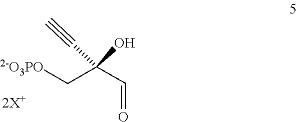
wherein 2X+ is (a) two protons, (b) one proton and one monovalent cation, (c) two monovalent cations wherein each said cation is the same or different, or (d) one divalent cation.

4. The compound 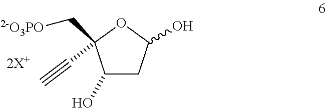
wherein 2X+ is (a) two protons, (b) one proton and one monovalent cation, (c) two monovalent cations wherein each said cation is the same or different, or (d) one divalent cation.

5. The compound 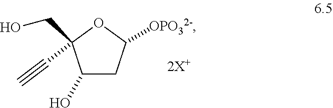
wherein 2X+ is (a) two protons. (b) one proton and one monovalent cation. (c) two monovalent cations wherein each said cation is the same or different, or (d) one divalent cation.


SEQUENCE LISTING (21 sequences)

SEQ ID NO: 1 - protein, 657 aa, Fusarium graminearum
MASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFEGSPGGITLTSSWDPSTGIVSDRTSTVTKHDMFCPGISMDGNGQIVVDETATGGNDAKKTSLYDSSSDSWIPGPDMQVARGYQSSATMSDGRVFTIGGSFSGGRVEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYEDSDATTNAHIITLGEPGTSPNTVFASNGLYFARTFHTSVVLPDGSTFITGGQRRGIPTEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQGGGGSWSHPQFEK

SEQ ID NO: 2 - protein, 316 aa, Escherichia coli
MSIKEQTLMTPYLQFDRNQWAALRDSVPMTLSEDEIARLKGINEDLSLEEVAEIYLPLSRLLNFYISSNLRRQAVLEQFLGTNGQRIPYIISIAGSVAVGKSTTARVLQALLSRWPEHRRVELITTDGFLHPNQVLKERGLMKKKGFPESYDMHRLVKFVSDLKSGVPNVTAPVYSHLIYDVIPDGDKTVVQPDILILEGLNVLQSGMDYPHDPHHVFVSDFVDFSIYVDAPEDLLQTWYINRFLKFREGAFTDPDSYFHNYAKLTKEEAIKTAMTIWKEMNWLNLKQNILPTRERASLILTKSANHAVEEVRLRK

SEQ ID NO: 3 - protein, 413 aa, Thermotoga maritima
MGSHHHHHHGSRVLVINSGSSSIKYQLIEMEGEKVLCKGIAERIGIEGSRLVHRVGDEKHVIERELPDHEEALKLILNTLVDEKLGVIKDLKEIDAVGHRVVHGGERFKESVLVDEEVLKAIEEVSPLAPLHNPANLMGIKAAMKLLPGVPNVAVFDTAFHQTIPQKAYLYAIPYEYYEKYKIRRYGFHGTSHRYVSKRAAEILGKKLEELKIITCHIGNGASVAAVKYGKCVDTSMGFTPLEGLVMGTRSGDLDPAIPFFIMEKEGISPQEMYDILNKKSGVYGLSKGFSSDMRDIEEAALKGDEWCKLVLEIYDYRIAKYIGAYAAAMNGVDAIVFTAGVGENSPITREDVCSYLEFLGVKLDKQKNEETIRGKEGIISTPDSRVKVLVVPTNEELMIARDTKEIVEKIGR

SEQ ID NO: 4 - protein, 613 aa, Streptococcus thermophilus
MGSSHHHHHHSSGLVPRGSHMTVGKTKVSTASLKVLAGWGIDTIYGIPSGTLAPLMEALGEQEETDIKFLQVKHEEVGAMAAVMQWKFGGKLGVCVGSGGPGASHLINGLYDAAMDNTPVLAILGSPPQRELNMDAFQELNQNPMYDHIAVYNRRVAYAEQLPKLIDDAIRTAISKRGVAVLEVPGDFGYKEIANDAFYSSGHSYRDYVSSAINEVDIDAAVEVLNKSKRAVIYAGIGTMGHGPAVQELSRKIKAPIITTAKNFETFDYDFEGLTGSTYRVGWKPANEAVKEADTVLFVGSNFPFAEVEGTFSNVENFIQIDNNPTMLGKRHNADVAILGDAGEAVQMLLEKVAPVEESAWWNANLKNIQNWRDYMTKLETKENGPLQLYQVYNAINKYADEDAIYSIDVGNTTQTSIRHLHMTPKNMWRTSPLFASMGIALPGGIGAKNVYPERQVFNLMGDGAFSMNYQDIVTNVRYNMPVINVVFTNTEYGFIKNKYEDTNTNTFGTEFTDVDYAMIGEAQGAVGFTVSRIEDMDQVMAAAVKANKEGKTVVIDAKITKDRPIPVETLKLDPALYSEEEIKAYKERYEAEELVPFSEFLKAEGLESKVAK

SEQ ID NO: 5 - protein, 257 aa, Shewanella halifaxensis
MSDLKKAAQQAISLMDLTTLNDDDTDQKVIELCHKAKTPAGDTAAICIYPRFIPIARKTLNEIGGDDIKIATVTNFPHGNDDIAIAVLETRAAVAYGADEVDVVFPYRALMEGNETVGFELVKACKEACGEDTILKVIIESGVLADPALIRKASELSIDAGADFIKTSTGKVAVNATLEAAEIMMTVISEKNPKVGFKPAGGVKDAAAAAEFLGVAARLLGDDWATPATFRFGASSLLTNLLHTLELADAPQGAQGY

SEQ ID NO: 6 - protein, 257 aa, Shewanella halifaxensis
MCDLKKAAQRAISLMDLTTLNDDDTDQKVIELCHKAKTPAGDTAAIVIYPRFIPIARKTLNEIGGLDIKIVTVTNFPHGNDDIAIAVLETRAAVAYGADEVDVVFPYRALMEGNETVGFELVKACKEACGEDTILKVIIESGVLKDPALIRKASEISIDAGADFIKTSTGKVAVNATLEAAEIIMTVISEKNPKVGFKPAGGIKDAAAAAEFLGVAARLLGDDWATPATFRFGATDLLTNLLHTLELADAPQGAQGY

SEQ ID NO: 7 - protein, 500 aa, Alloscardovia omnicolens
MKNKVQLITYADRLGDGTLKSMTETLRKHFEGVYEGVHILPFFTPFDGADAGFDPVDHTKVDPRLGSWDDVAELSTTHDIMVDTIVNHMSWESEQFQDVMAKGEDSEYYPMFLTMSSIFPDGVTEEDLTAIYRPRPGLPFTHYNWGGKTRLVWTTFTPQQVDIDTDSEMGWNYLLSILDQLSQSHVSQIRLDAVGYGAKEKNSSCFMTPKTFKLIERIKAEGEKRGLETLIEVHSYYKKQVEIASKVDRVYDFAIPGLLLHALEFGKTDALAQWIDVRPNNAVNVLDTHDGIGVIDIGSDQMDRSLAGLVPDEEVDALVESIHRNSKGESQEATGAAASNLDLYQVNCTYYAALGSDDQKYIAARAVQFFMPGVPQVYYVGALAGSNDMDLLKRTNVGRDINRHYYSAAEVASEVERPVVQALNALGRFRNTLSAFDGEFSYSNADGVLTMTWADDATRATLTFAPKANSNGASVARLEWTDAAGEHATDDLIANPPVVA

SEQ ID NO: 8 - protein, 407 aa, Escherichia coli
MKRAFIMVLDSFGIGATEDAERFGDVGADTLGHIAEACAKGEADNGRKGPLNLPNLTRLGLAKAHEGSTGFIPAGMDGNAEVIGAYAWAHEMSSGKDSVSGHWEIAGVPVLFEWGYFSDHENSFPQELLDKLVERANLPGYLGNCRSSGTVILDQLGEEHMKTGKPIFYTSAASVFQIACHEETFGLDKLYELCEIAREELTNGGYNIGRVIARPFIGDKAGNFQRTGNRRDLAVEPPAPTVLQKLVDEKHGQVVSVGKIADIYANCGITKKVKATGLDALFDATIKEMKEAGDNTIVFTNFVDFDSSWGHRRDVAGYAAGLELFDRRLPELMSLLRDDDILILTADHGCDPTWTGTDHTREHIPVLVYGPKVKPGSLGHRETFADIGQTLAKYFGTSDMEYGKAMF

SEQ ID NO: 9 - protein, 239 aa, Escherichia coli
MATPHINAEMGDFADVVLMPGDPLRAKYIAETFLEDAREVNNVRGMLGFTGTYKGRKISVMGHGAGIPSCSIYTKELITDFGVKKIIRVGSCGAVLPHVKLRDVVIGMGACTDSKVNRIRFKDHDFAAIADFDMVRNAVDAAKALGIDARVGNLFSADLFYSPDGEMFDVMEKYGILGVEMEAAGIYGVAAEFGAKALTICTVSDHIRTHEQTTAAERQTTFNDMIKIALESVLLGDKE

SEQ ID NO: 10 - protein, 650 aa, Fusarium graminearum
MASAPIGVAIPRNNWAVTCDSAQSGNECNKAIDGNKDTFWHTQYGVNGDPKPPHTITIDMKTVQNVNGLSVLPRQDGNQNGWIGRHEVYLSSDGVNWGSPVASGSWFADSTTKYSNFETRPARYVRLVAITEANGQPWTSIAEINVFQASSYTAPQPGLGRWGPTIDLPIVPSAAAIEPTSGRVLMWSSYRQDAFEGSPGGITLTSSWDPSTGIVSDRTSTVTKHDMFCPGISMDGNGQIVVSGGNDAKKTSLYDSSSDSWIPGPDMQVARGYQSSATMSDGRVFTIGGSFSGGQVEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYEDSDATTNAHIITLGEPGTSPNTVFASNGLYFARTFHTSVVLPDGSTFITGGQQRGIPTEDSTPVFTPEIYVPEQDTFYKQNPNSIVRAYHSISLLLPDGRVFNGGGGLCGDCTTNHFDAQIFTPNYLYDSNGNLATRPKITRTSTQSVVVGGWITIWTDMSISAASLIRYGTATHTVNTDQRRIPLTLTNNGGNSYSFQVPSDSGVALPGYWMLFVMNSAGVPSVASTIRVTQGQTGHHHHHH

SEQ ID NO: 11 - protein, 650 aa, Fusarium graminearum
MASAPIGVAIPRNNWAVTCDSAQSGNECNKAIDGNKDTFWHTQYGVNGDPKPPHTITIDMKTVQNVNGLSVLPRQDGNQNGWIGRHEVYLSSDGVNWGSPVASGSWFADSTTKYSNFETRPARYVRLVAITEANGQPWTSIAEINVFQASSYTAPQPGLGRWGPTIDLPIVPSAAAIEPTSGRVLMWSSYRQDAFEGSPGGITLTSSWDPSTGIVSDRTSTVTGHDMFCPGISMDGNGQIVVSGGNDAKKTSLYDSSSDSWIPGPDMQVARGYNSSATMSDGRVFTIGGSFSGGQVEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLLFARTFHTSVVLPDGSTFITGGQQRGIPTEDSTPVFTPEIYVPEQDTFYKQNPNSIVRAYHSISLLLPDGRVFNGGGGLCGDCETNHFDAQIFTPNYLYDSNGNLATRPKITRTSTQSVVVGGWITIWTDMSISAASLIRYGTATHTVNTDQRRIPLTLTNNGGNSYSFQVPSDSGVALPGYWMLFVMNSAGVPSVASTINVTQGQTGHHHHHH

SEQ ID NO: 12 - protein, 316 aa, Escherichia coli
MSIKEQTLMTPYLQLDRNQWAALRDSNPMTLSEDEIARLKGINEDLSLEEVAEVYLPLSRLLNFYISSNLRRQAVLEQFLGTNGQRIPYIISIAGSVAVGKSTTARVLQALLSRWPEHRRVELITTDGFLHPNQVLKERGLMKKKGFPESYDMHRLMKFVKDLKSGVPNVTAPVYSHLIYDVIPDGDKTVVQPDILILEGLNVLQSGMDYPHDPHHVFVSDFVDFSIYVDAPEDLLQTWYINRFLKFREGAFTDPDSYFHGYAKLTKEEAIKTAMTIWKEMNHLNLKQNILPTRERASLILTKSANHIVEEVRLRK

SEQ ID NO: 13 - protein, 325 aa, Escherichia coli
MHHHHHHGGMSIKEQTLMTPYLQLDRNQWAALRDSNPMTLSEDEIARLKGINEDLSLEEVAEVYLPLSRLLNFYISSNLRRQAVLEQFLGTNGQRIPYIISIAGSVAVGKSTTARVLQALLSRWPEHRRVEHITTDGFLHPNQVLKERGLMGKKGFPESYDMHRLMKFVKDLKSGVPNVTAPVYSHLIYDVIPDGDKTVVQPDILILEGLNVLQSGMDYPHDPHHVFVSDFVDFSIYVDAPEDLLQTWYINRFLKFREGAFTDPDSYFHGYAKLTKEEAIKTAMTIWKEMNHLNLKQNILPTRERASLILTKSANHIVEEVRLRK

SEQ ID NO: 14 - protein, 263 aa, Shewanella halifaxensis
MHHHHHHCDLKKAAQRAISLMDLTTLNDDDTDQKVIELCHKAKTPAGDTAAIVIYPRFIPIARKTLNEIGGLDIKIVTVTNFPHGNDDIAIAVLETRAAVAYGADEVDVVFPYRALMEGNETVGFELVKACKEACGEDTILKVIIESGVLKDPALIRKASEISIDAGADFIKTSTGKVAVNATLEAAEIIMTVISEKNPKVGFKPAGGIKDAAAAAEFLGVAARLLGDDWATPATFRFGATDLLTNLLHTLELADAPQGAQGY

SEQ ID NO: 15 - protein, 239 aa, Escherichia coli
MATPHINAEMGDFADVVLMPGDPLRAKYIAETFLEDAREVNNVRGMLGFTGTYKGRKISVMGHGMGIPSCSIYTKELITDFGVKKIIRVGSCGAVLPHVKLRDVVIGMGACTDSKVNRIRFKDHDFAAIADFDMVRNAVDAAKALGIDARVGNLFSADLFYSPDGEMFDVMEKYGILGVEMEAAGIYGVAAEFGAKALTICTVSDHIRTHEQTTAAERQTTFNDMIKIALESVLLGDKE

SEQ ID NO: 16 - protein, 650 aa, Fusarium graminearum
MASAPIGVAIPRNNWAVTCDSAQSGNECNKAIDGNKDTFWHTQYGVNGDPKPPHTITIDMKTVQNVNGLSVLPRQDGNQNGWIGRHEVYLSSDGVNWGSPVASGSWFADSTTKYSNFETRPARYVRLVAITEANGQPWTSIAEINVFQASSYTAPQPGLGRWGPTIDLPIVPSAAAIEPTSGRVLMWSSYRQDAFEPSPGGITLTSSWDPSTGIVSDRTSTVTGHDMFCPGISMDGNGQIVVSGGNDAKKTSLYDSSSDSWIPGPDMQVARGYNSSATMSDGRVFTIGGSYSGGQVEKNGEVYSPSSKTWTSLPNAKVNPMLTADKQGLYRSDNHAWLFGWKKGSVFQAGPSTAMNWYYTSGSGDVKSAGKRQSDRGVAPDAMCGNAVMYDAVKGKILTFGGSPDYQDSDATTNAHIITLGEPGTSPNTVFASNGLLFARTFHTSVVLPDGSVFITGGQQRGVPLEDSTPVFTPEIYVPEQDTFYKQNPNSIVRAYHSISLLLPDGRVFNGGGGLCGDCETNHFDAQIFTPNYLYDSNGNLATRPKITRTSTQSVVVGGWITIWTDMSISAASLIRYGTATHTVNTDQRRIGLTLTNNGGNSYSFQVPSDSGVALPGYWMLFVMNSAGVPSVASTINVTQGQTGHHHHHH

SEQ ID NO: 17 - protein, 650 aa, Fusarium graminearum
MASAPIGVAIPRNNWAVTCDSAQSGNECIKAIDGNKDTFWHTQYGVNGDPKPPHTITIDMKTVQNVNGLSVLPRQDGNQNGWIGRHEVYLSSDGVNWGSPVASGSWFADSTTKYSNFETRPARYVRLVAITEANGQPWTSIAEINVFQASSYTAPQPGLGRWGPTIDLPIVPSAAAIEPTSGRVLMWSSYRQDAFEDSPGGITLTSSWDPSTGIVSDRTSTVTGHDMFCPGISMDGNGQIVVSGGNDAKKTSLYDSSSDSWIPGPDMQVARGYNSSATMSDGRVFTIGGSYSGGQVEKNGEVYSPSSKTWTSLPNAKVNPMLTADKQGLYRSDNHAWLFGWKKGSVFQAGPSTAMNWYYTSGSGDVKSAGKRQSDRGVAPDAMCGNAVMYDAVKGKILTFGGSPDYQDSDATTNAHIITLGEPGTSPNTVFASNGLLFARTFHTSVVLPDGSVFITGGQQRGVPLEDSTPVFTPEIYVPEQDTFYKQNPNSIVRAYHSISLLLPDGRVFNGGGGLCGDCETNHFDAQIFTPNYLYDSNGNLATRPKITRTSTQSVVVGGWITIWTDMSISAASLIRYGTATHTVNTDQRRIGLTLTNNGGNSYSFQVPSDSGVALPGYWMLFVMNSAGVPSVASTINVTQGQTGHHHHHH

SEQ ID NO: 18 - protein, 650 aa, Fusarium graminearum
MASAPIGVAIPRNNWAVTCDSAQSGNECIKAIDGNKDTFWHTQYGVNGDPKPPHTITIDMKTVQNVNGLSVLPRQDGNQNGWIGRHEVYLSSDGVNWGSPVASGSWFADSTTKYSNFETRPARYVRLVAITEANGQPWTSIAEINVFQASSYTAPQPGLGRWGPTIDLPIVPSAAAIEPTSGRVLMWSSYRQDAFEDSPGGITLTSSWDPSTGIVSDRTSTVTGHDMFCPGISMDGNGQIVVSGGNDAKKTSLYDSSSDSWIPGPDMQVARGYNSSATMSDGRVFTIGGSYSGGQVEKNGEVYSPSSKTWTSLPNAKVNPMLTADKRGLYRSDNHAWLFGWKKGSVFQAGPSTAMNWYYTSGSGDVKSAGKRQSDRGVAPDAMCGNAVMYDAVKGKILTFGGSPDYQDSDATTNAHIITLGEPGTSPNTVFASNGLLFARTFHTSVVLPDGSVFITGGQQRGVPLEDSTPVFTPEIYVPEQDTFYKQNPNSIVRAYHSISLLLPDGRVFNGGGGLCGDCETNHFDAQIFTPNYLYDSNGNLATRPKITRTSTQSVVVGGWITIWTDMSISAASLIRYGTATHTVNTDQRRIGLTLTNNGGNSYSFQVPSDSGVALPGYWMLFVMNSAGVPSVASTINVTQGQTGHHHHHH

SEQ ID NO: 19 - protein, 650 aa, Fusarium graminearum
MASAPIGVAIPRNNWAVTCDSAQSGNECIKAIDGNKDTFWHTQYGVNGDPKPPHTITIDMKTVQNVNGLSVLPRQDGNQNGWIGRHEVYLSSDGVNWGSPVASGSWFADSTTKYSNFETRPARYVRLVAITEANGQPWTSIAEINVFQASSYTAPQPGLGRWGPTIDLPIVPSAAAIEPTSGRVLMWSSYRQDAFRDSPGGITLTSSWDPSTGIVSDRTSTVTGHDMFCPGISMDGNGQIVVSGGNDAKKTSLYDSSSDSWIPGPDMQVARGYNSSATMSDGRVFTIGGSYSGGQVEKNGEVYSPSSKTWTSLPNAKVNPMLTADKQGLYRSDNHAWLFGWKKGSVFQAGPSTAMNWYYTSGSGDVKSAGKRQSDRGVAPDAMCGNAVMYDAVKGKILTFGGSPDYQDSDATTNAHIITLGEPGTSPNTVFASNGLLFARTFHTSVVLPDGSVFITGGQQRGVPLEDSTPVFTPEIYVPEQDTFYKQNPNSIVRAYHSISLLLPDGRVFNGGGGLCGDCETNHFDAQIFTPNYLYDSNGNLATRPKITRTSTQSVVVGGWITIWTDMSISAASLIRYGTATHTVNTDQRRIGLTLTNNGGNSYSFQVPSDSGVALPGYWMLFVMNSAGVPSVASTINVTQGQTGHHHHHH

SEQ ID NO: 20 - protein, 326 aa, Escherichia coli
MHHHHHHGGSGSIKEQTLMTPYLQLDRNQWAALRDSNPMTLSEDEIARLKGINEDLSLEEVAEVYLPLSRLLNFYISSNLRRQAQLEQFLGTNGQRIPYIISIAGSVAVGKSTFARVLQALLSRWPEHRRVEHITTDGFLHPNQVLKERGLMGKKGFPESYDMHRLMKFVKDLKSGVPNVTAPVYSHLIYDVIPDGDKTVVQPDILILEGLNVLQSGMDYPHDPHHVFVSDFVDFSIYVDAPEDLLQTWYINRFLKFREGAFTDPDSYFHGYAKLTKEEAIKTAMTIWKEMNHVNLKQNILPTRERASLILTKSANHIVEEVRLRK

SEQ ID NO: 21 - protein, 413 aa, Thermotoga maritima
MGSHHHHHHGSRVLNINSGSSSIKYQLIEMEGEKVLCKGIAERIGIEGSRLVHRVGDEKHVIERELPDHEEALKLILNTLVDEKLGVIKDLKEIDAVGHRVVHGGERFKESVLVDEEVLKAIEEVSPLAPLHNPANLMGIKAAMKLLPGVPNVQVFDTAFHQTIPQKAYLYAIPYEYYEKYKIRRYGFHGISHRYVSKRAAEILGKKLEELKIITCHIGNGASVAAVKYGKCVDTSMGFTPLEGLVMGTRSGDLDPAIPFFIMEKEGISPQEMYDILNKKSGVYGLSKGFSSDMRDNLEAALKGDEWCKLVLEIYDYRIAKYIGAYAAAMNGVDAIVFTAGVGENSPITREDVCKYLEFLGVKLDKQKNEETIRGKEGIISTPDSRVKVLVVPTNEELMIARDTKEIVEKIGR